(19) United States
(12) Patent Application Publication
(10) Pub. No.: US 2026/0132443 A1
(43) Pub. Date: May 14, 2026

(54) Long Stokes Shift Conjugated Polymers for Next Generation Sequencing

(71) Applicant: Illumina, Inc., San Diego, CA (US)

(72) Inventors: Iuliana Petruta Maria, Cambridge (GB); Nam Trong Nguyen, Cambridge (GB); Dale Weekes, Haverhill (GB); Carole Anastasi, Babraham (GB); Xavier von Hatten, Cambridge (GB); Wayne N. George, Waltham Cross (GB); Andrew A. Brown, Cambridge (GB)

(73) Assignee: Illumina, Inc., San Diego, CA (US)

(21) Appl. No.: 19/386,422

(22) Filed: Nov. 12, 2025

Related U.S. Application Data
(60) Provisional application No. 63/719,836, filed on Nov. 13, 2024.

(51) Int. Cl. C12Q 1/6818; C12Q 1/6874

(52) U.S. Cl. CPC C12Q 1/6818; C12Q 1/6874

(57) ABSTRACT

The disclosure concerns methods of high throughput sequencing that utilize labeled moieties comprising a fluorophore having a Stokes Shift of at least 90 nm.

### CROSS REFERENCE TO RELATED APPLICATIONS

This application claims the benefit of U.S. Provisional Application Patent Application No. 63/719,836, filed Nov. 13, 2024, which is incorporated herein by reference in its entirety.

### BACKGROUND

The technology disclosed relates to fluorophores for supporting optics-based biological and chemical assay protocols in various genomic, exogenomic, transcriptomic, and proteomic domains, including, e.g., high throughput screening (HTP), nucleic acid sequencing implemented using Next Generation Sequencing (NGS), variant detection and gene expression analyses, and the like. By way of example, sequencing methodologies for nucleic acid materials on NGS platforms commonly deploy deoxyribonucleic acid (DNA) libraries in which a DNA target (e.g., genomic DNA (gDNA), or complimentary DNA (cDNA)) is processed into fragments and ligated with technology-specific adaptors. NGS workflow using, e.g., a sequence-by-synthesis (SBS) technique, involves loading a DNA library onto a flow cell and hybridizing individual DNA fragments to adapter-specific complimentary oligonucleotides (oligos) covalently bound to a solid support surface of the flow cell; clustering the individual fragments into thousands of identical DNA template strands (amplicons) through bridge or exclusion amplification; and, finally, sequencing, in which copy strands are simultaneously synthesized and sequenced on the DNA templates using a reversible terminator-based process that detects signals emitted from fluorophore-labeled single bases as they are added round by round to the copy strands. Because the multiple template strands of each cluster have the same sequence, base pairs incorporated into the corresponding copy strands in each round will be the same, and thus the signal generated from each round will be enhanced proportional to the number of copies of the template strand in the cluster.

NGS operates on massively parallel multiplex platforms that can process sequencing volumes of nucleotides in the billions within very short runtimes and at low cost. For example, Illumina's NovaSeq 6000 sequencing system can generate output in the range of 1.6-40 billion paired end reads at a run time ranging between 13 and 44 hours. By comparison, the Human Genome Project, which sequenced the first human genome using capillary sequencing, took around 10 years.

Despite vast improvements in output volume, run times, and cost effectiveness, certain operating constraints persist that lock out additional gains in processing efficiency. As just one example, a typical step and shoot sequencing operation using a flow cell format and 300 cycle SBS chemistry may obtain two images per sequencing cycle per tile using two-exposure (2×), two-channel chemistry, where the total number of cycles is equal to the length in bp of the DNA template strand, viz., 300 bp, and the total number of tiles image may be 130 or greater, resulting in a total number of imaging steps that can exceed 78,000 (130×300×2). Accordingly, any reduction in the number of images required per cycle would reduce power consumption and increase operational efficiency. In addition, fewer images would reduce potential damage to the nucleic acid material caused by the laser exposure.

### SUMMARY

The present disclosure relates to fluorophores having increased Stokes shift compared to present compounds. Fluorophores herein enable one exposure (1×) two-channel chemistry with minimal crosstalk between channels. Step and shoot sequencing operations using 1×, two channel chemistry enabled by fluorophores of the present disclosure can reduce the total number of imaging steps by half, e.g., 39,000, using a flow cell format and 300 cycle SBS chemistry.

In some aspects, the disclosure concerns methods of fluorescently detecting a polynucleotide material, which can include the steps of (a) providing a substantially monoclonal polynucleotide analyte immobilized on a solid support surface; (b) contacting the analyte with a one-exposure, two channel reagent solution, wherein the reagent solution can be a mixture of nucleosides, each nucleoside comprising a first-type, second-type, third-type or fourth-type nucleobase, a polymerase, and a primer, wherein nucleosides comprising the first type nucleobase can be labeled with a blue-blue fluorophore, nucleosides comprising the second type nucleobase can be labeled with a blue-green fluorophore having the following structure:

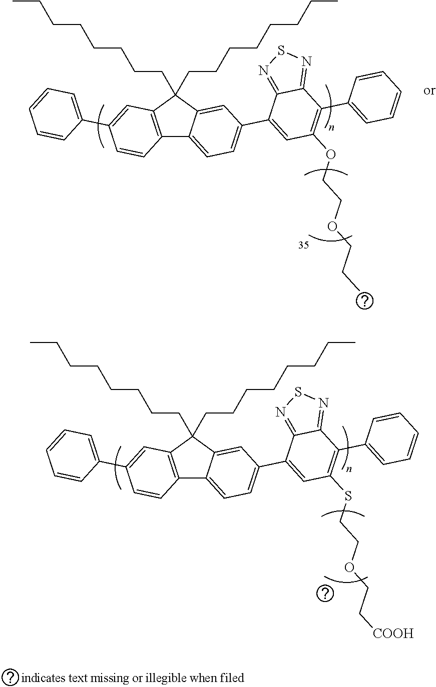

wherein n is 1 to about 1000; wherein the conjugated polymer nanoparticle has a Stokes shift of at least 90 nm, wherein nucleosides with the third-type nucleobase can be labeled with the blue-blue fluorophore and the blue-green fluorophore or the third-type nucleobase can be labeled with a fluorophore having an emission between blue emission of the first-type nucleobase and the green emission of the second type nucleobase, and the nucleosides with the fourth-type nucleobase can be unlabeled; and wherein one of the nucleosides is configured to hybridize to the analyte; (c) exposing the analyte to excitation in a blue channel; and (d) detecting a fluorescent signal in the analyte; wherein a blue channel signal indicates a first-type nucleoside hybridized to the analyte, a green channel signal indicates a second-type nucleoside, a signal detectable in both the blue and green channels indicates a third-type nucleoside, and no signal indicates a fourth-type nucleoside.

In some embodiments, the Stokes shift of the blue-green fluorophore can be at least 90 nm or 100 nm or about 100 nm to about 120 nm.

In certain embodiments, the first, second, third and fourth-type nucleosides can be selected from adenosine (A), cytosine (C), guanosine (G) and thymidine (T) such that each of the first, second, third and fourth-type nucleosides are a different nucleoside. In other embodiments, the first, second, third and fourth-type nucleosides can be selected from adenosine (A), cytosine (C), guanosine (G) and uracil (U) such that each of the first, second, third and fourth-type nucleosides are a different nucleoside.

In some embodiments, the solid support surface can include a base layer, an optic and chemical support layer of cured resin material adhered to the support layer, wherein the optic and chemical support layer comprises an array of depressions or nanowells separated by interstitial regions of featureless resin surface, each array comprising: a plurality of nanowells impressed in the optic and chemical support layer, wherein the nanowells define openings coplanar with the featureless resin surface, the nanowells comprising a base portion providing a solid support for optic detection of the polynucleotide analyte, a reaction site collocated with at least a portion of the solid support, a conformal shell of high refractive index medium supporting the interior volume of the cavity, and wherein the surface chemistry is localized to the reaction site and comprises a capture agent configured to interact with a constituent analyte of the polynucleotide material.

The optic and chemical support layer can comprise a resin that serves as not only the “optic support layer,” but the resin also serves as a “chemistry support layer” upon which the cluster will eventually be grown and sequenced.

In some embodiments, the blue-green fluorophore can be a nanoparticle. In certain embodiments, fluorophore can have an emission between blue emission of the first-type nucleobase and the green emission of the second type nucleobase can be a nanoparticle. In some embodiments, the nanoparticle can have a size of from about 1 nm and 500 nm.

In other aspects, the disclosure concern methods of fluorescently detecting a polynucleotide material, the method comprising providing a substantially monoclonal polynucleotide analyte immobilized on a solid support surface; contacting the analyte with a one-exposure, two channel reagent solution, wherein the reagent solution comprises a mixture of nucleosides, each nucleoside comprising a first-type, second-type, third-type or fourth-type nucleobase, a polymerase, and a primer, wherein nucleosides comprising the first type nucleobase are labeled with a blue-blue fluorophore, nucleosides comprising the second type nucleobase are labeled with a blue-green fluorophore having a Stokes shift of at least 90 nm, nucleosides with the third-type nucleobase are labeled with the blue-blue fluorophore and the blue-green fluorophore or the third-type nucleobase can be labeled with a fluorophore having an emission between blue emission of the first-type nucleobase and the green emission of the second type nucleobase, and the nucleosides with the fourth-type nucleobase is unlabeled; and wherein one of the nucleosides is configured to hybridize to the analyte; exposing the analyte to excitation in a blue channel; and detecting a fluorescent signal in the analyte; wherein a blue channel signal indicates a first-type nucleoside hybridized to the analyte, a green channel signal indicates a second-type nucleoside, a signal detectable in both the blue and green channels indicates a third-type nucleoside, and no signal indicates a fourth-type nucleoside.

In some embodiments, at least one of the fluorophores has one of the following structures:

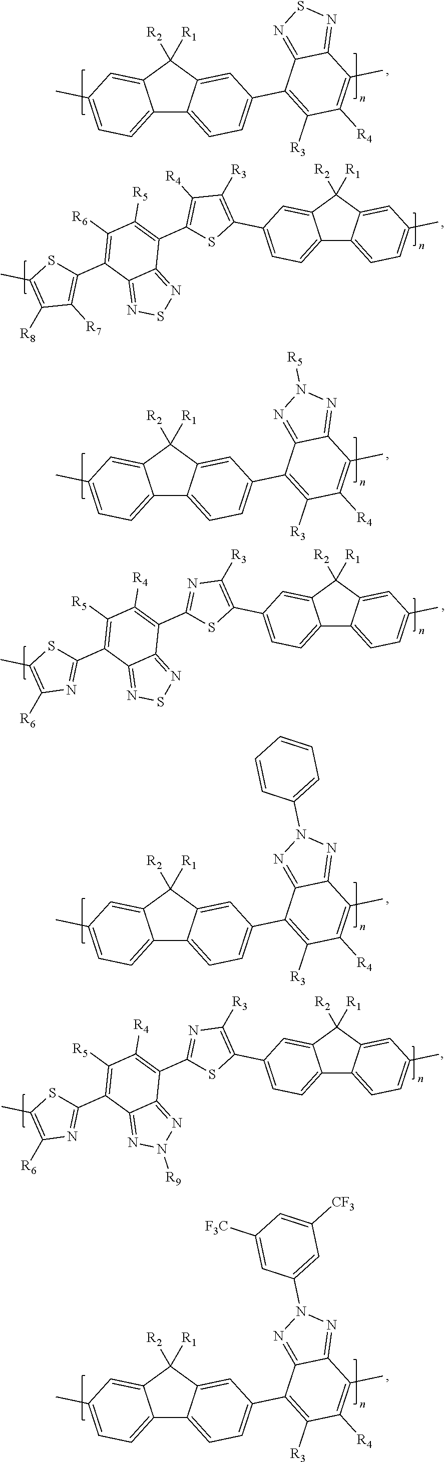

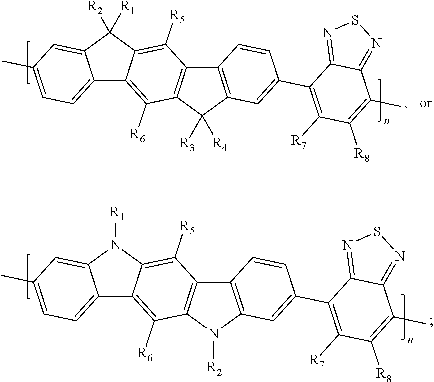

wherein n is 1 to about 1000; and wherein each R₁-R₉ is independently H, C₁-C₁₂ alkyl, substituted or unsubstituted aryl, heteroaryl, dialkyl amine, diaryl amine, substituted or unsubstituted ether, substituted or unsubstituted polyether, halogen, or polyethylene glycol chains having 1 to 50 units.

Some polyethylene glycol chains can be terminated with azide, tetrazine, dibenzocyclooctyne (DBCO), bicyclo(6.1.0)non-4-yne (BCN), trans-cyclooctene (TCO), streptavidin, carboxylic acid, or an amine. In yet other embodiments, R₁-R₉ can include polyacrylamide (PAAm), polyvinyl pyrrolidone (PVP), polyvinyl alcohol (PVA), polyacrylic acid, poly N-2-Hydroxypropyl methacylamide (PHPMA), polyoxazolines, and the like.

In some embodiments, the Stokes shift of the blue-green fluorophore can be at least 100 nm or about 100 nm to about 120 nm.

In certain embodiments, the first, second, third and fourth-type nucleosides can be selected from adenosine (A), cytosine (C), guanosine (G) and thymidine (T) such that each of the first, second, third and fourth-type nucleosides are a different nucleoside. In other embodiments, the first, second, third and fourth-type nucleosides can be selected from adenosine (A), cytosine (C), guanosine (G) and uracil (U) such that each of the first, second, third and fourth-type nucleosides are a different nucleoside.

In some embodiments, the solid support surface can include a base layer, an optic support layer of cured resin material adhered to the support layer, wherein the optic support layer comprises an array separated by interstitial regions of featureless resin surface, each array comprising: a plurality of nanowells impressed in the optic support layer, wherein the nanowells define openings coplanar with the featureless resin surface, the nanowells comprising a base portion providing a solid support for optic detection of the polynucleotide analyte, a reaction site collocated with at least a portion of the solid support, and wherein the surface chemistry is localized to the reaction site and comprises a capture agent configured to interact with a constituent analyte of the polynucleotide material.

In some embodiments, the blue-green fluorophore can be a nanoparticle. In yet other aspects, the disclosure provides a one exposure, two channel reagent kit that can include: a mixture of nucleosides in solution, each nucleoside comprising a first-type, second-type, third-type or fourth-type nucleobase; a polymerase; and a primer; wherein nucleosides comprising the first type nucleobase are labeled with a blue-blue fluorophore, nucleosides comprising the second type nucleobase are labeled with a blue-green chromophore, nucleosides with the third-type nucleobase are labeled with the blue-blue fluorophore and the blue-green fluorophore and the nucleosides or the third-type nucleobase can be labeled with a fluorophore having an emission between blue emission of the first-type nucleobase and the green emission of the second type nucleobase, and nucleotides comprising the fourth-type nucleobase can be unlabeled.

In some kits, the Stokes shift of the blue-green fluorophore can be at least 100 nm.

In certain embodiments, the first, second, third and fourth-type nucleosides can be selected from adenosine (A), cytosine (C), guanosine (G) and thymidine (T) such that each of the first, second, third and fourth-type nucleosides are a different nucleoside. In other embodiments, the first, second, third and fourth-type nucleosides can be selected from adenosine (A), cytosine (C), guanosine (G) and uracil (U) such that each of the first, second, third and fourth-type nucleosides are a different nucleoside.

### BRIEF DESCRIPTION OF THE DRAWINGS

FIG. 1A is a top view of an example flow cell.

FIG. 1B is an enlarged, and partially cutaway view of an example flow cell architecture and functionalized solid support of the flow cell of FIG. 1A.

FIG. 2 illustrates an example two-channel chemistry that can be used with NGS.

FIGS. 3A-3C show, in FIG. 3A, expected optical properties of a fluorophore set used for 1Ex-2Ch detection. The fluorophores should show similar excitation spectra (dotted line) and allow excitation at a single wavelength. A long Stokes shift fluorophore and a short/standard Stokes shift fluorophore allows emission across two different channels, while a medium Stokes shift fluorophore with broad emission in both channels can act as the “corner” fully functional nucleotides (FFN). FIGS. 3B and 3C show examples of options to introduce conjugated polymers (CPs) for 1Ex-2Ch detection.

FIGS. 4A and 4B show expected optical properties of a fluorophore set used for 1Ex-3Ch detection. In FIG. 4A, the fluorophores should show similar excitation spectra (dotted line) and allow excitation at a single wavelength and show different Stokes shifts that allow emission (solid line) across three different channels. FIG. 4B shows examples of options to introduce CPs for 1Ex-3Ch detection.

FIG. 5 presents a literature example of 1Ex-3Ch conjugated polymers with narrow emission profiles using BODIPY acceptor dyes. Source: Anal. Chem. 2017, 89, 6232-6238.

FIGS. 6A and 6B show, in FIG. 6A, synthesis of CP-F, CP-PEG36-N3 and CP-PEG12-COOH. FIG. 6B shows formation of functionalized conjugated polymer nucleotides (CPNs) from the conjugated polymers.

FIG. 7 presents excitation (dotted line) and emission (solid line) spectra of CP-PEG36-N3 (left) and CP-PEG12-COOH (right) nanoparticles in water.

FIG. 8 presents a plot of fluorescence intensity vs. concentration for NR550-C4-COOH (left) and CP-PEG36-N3 nanoparticles (right) in scan mix.

FIGS. 9A-9C shows scatterplots obtained, in FIG. 9A, with a standard incorporation mixture containing ffG, ffC-G2-NR550S0, ffT-G2-MC485CQ-O-COT and ffA-G2-BL-NR455Boc; in FIGS. 9B and 9C, a second incorporation mix containing ffG, ffC-G2-NR550S0, ffA-G2-BL-NR455Boc and an unlabeled FFT having a reactant DBCO moiety.

FIGS. 10A-10C presents scatterplots obtained, in FIG. 10A, with a standard incorporation mixture containing ffG, ffC-G2-NR550S0, ffT-G2-MC485CQ-O-COT and ffA-G2-BL-NR455Boc; in FIGS. 10B and 10C, a second incorporation mix containing ffG, ffC-G2-NR550S0, ffT-G2-Vega and an unlabeled FFA having a reactant biotin moiety.

### DETAILED DESCRIPTION

Base detection using a single excitation-two emission (1Ex-2Ch) scheme is highly attractive for future detection platforms due to reduced laser costs and simplified fully functionalized nucleotides (FFN) design. 3-Channel (2Ex-3Ch or 1Ex-3Ch) detection is also a promising strategy to maximize signal and signal to noise ratio (SNR) by removing the need to attenuate the brightness of FFNs and match the intensity of the corner cloud. An additional advantage for a 1Ex-2Ch scheme is that it allows for less laser exposure that may damage the nucleic acid sample that is being analyzed.

The ability to implement these detection schemes relies on having access to fluorophores with much longer Stokes shift than standard state of the art dyes. Fluorophores with large and tunable Stokes shifts have the ability to minimize crosstalk between channels, as well as reduce the overlap between their excitation and emission spectra, which may lead to self-quenching. However, most widely adopted organic dyes (fluorescein, rhodamine, cyanine, etc.) show short Stokes shifts of <30 nm and require significant structure engineering to increase them.

Conjugated polymers (CPs) are attractive fluorophores for base detection and SBS chemistry due to their stronger light-harvesting ability, higher brightness per volume and enhanced photostability compared with small molecule organic dyes.

As disclosed herein, use of conjugated polymers and conjugated polymer nanoparticles as long Stokes shift fluorophores for base labeling using 1Ex-2Ch, 2Ex-3Ch and 1Ex-3Ch detection schemes is possible. In addition to their brightness and photostability benefits, many CPs feature strong intramolecular charge transfer (ICT) and longer Stokes shifts than standard organic dyes, without the need for complex structure engineering. It is demonstrated that an example CPN is ˜1400× brighter in solution compared to Illumina commercial green dyes and can be used as a blue excitation-green emission fluorophore using both covalent and non-covalent post-incorporation labeling (PIL) of bases during sequencing-by synthesis (SBS). This strategy can also be implemented with water-soluble conjugated oligomers or polymers, in addition to nanoparticles, using either PIL or conventional FFN incorporation.

As used herein, a “blue-blue fluorophore” is a fluorophore that absorbs blue light and emits blue light. A “blue-green fluorophore,” such as the conjugated polymers disclosed herein, is a fluorophore that absorbs blue light and emits green light. A fluorophore that has both blue-blue and blue-green fluorophores emits light detectable in both the blue and green channels. A nucleobase that does not comprise a fluorophore may be detected by an absence of fluorescence. In some embodiments, blue excitation for each of the FFNs (first, second, and third nucleotides) results in emission in short Stokes shift in blue (first-type nucleobase), long Stokes shift emission in green (second-type nucleobase) and broad emission (third-type nucleobase emits) in both the green and blue channel.

In some embodiments, the sample may be a DNA sample where the first, second, third and fourth-type nucleosides can be selected from adenosine (A), cytosine (C), guanosine (G) and thymidine (T) such that each of the first, second, third and fourth-type nucleosides are a different nucleoside. In other embodiments, the sample can be an RNA sample where the first, second, third and fourth-type nucleosides can be selected from adenosine (A), cytosine (C), guanosine (G) and uracil (U) such that each of the first, second, third and fourth-type nucleosides are a different nucleoside.

As used herein, an “optic signal,” “output signal,” “emission signal,” or “signal” refers to a detectable event such as an emission, such as light emission, for example, in an image. Thus, in some implementations, a signal may represent any detectable light emission that is captured in an image (i.e., a “spot”) from a fluorescing analyte (as a “point source”). Thus, as used herein, “signal” may refer to both an actual emission from an analyte of the specimen and may refer to a spurious emission that does not correlate to an actual analyte. Thus, a signal could arise from noise and could be later discarded as not representative of an actual analyte of a specimen. A signal consistent with the disclosure includes, for example, fluorescent, luminescent, scatter, or absorption signals. Signals can be detected in the ultraviolet (UV) range (about 200 to 390 nm), visible (VIS) range (about 391 to 770 nm), infrared (IR) range (about 0.771 to 25 microns), or other ranges of the electromagnetic spectrum. Signals may be detected in a way that excludes all or part of one or more of these ranges.

A signal may include light emissions from conjugate fluorescent species of a biological material or chemical reagent, such as those discussed herein. A signal may also include transmitted light refracted and/or reflected by optical substrates. Optical signals, including excitation radiation that is incident upon the sample and light emissions that are provided by the sample, may have one or more spectral patterns. For example, more than one type of fluorescent species may be excited in an imaging session. In such cases, the different types of fluorescent species may be excited by a common excitation light source or may be excited by different excitation light sources that simultaneously provide incident light. Each type of fluorescent species may emit optical signals having a spectral pattern that is different from the spectral pattern of other labels. For example, the spectral patterns may have different emission spectra. The light emissions may be filtered to separately detect the optical signals from other emission spectra. As used herein, when the term “different” is used with respect to emission spectra, the emission spectra may have wavelength ranges that at least partially overlap so long as at least a portion of one emission spectrum does not completely overlap the other emission spectrum. Different emission spectra may have other characteristics that do not overlap, such as emission anisotropy or fluorescence lifetime. When the light emissions are filtered, the wavelength ranges of the emission spectra may be narrowed.

As used herein, the term “signal level” is intended to mean an amount or quantity of detected energy or coded information that has a desired or predefined characteristic. For example, an optical signal may be quantified by one or more of intensity, SNR, wavelength, energy, frequency, power, luminance, or the like. Other signals may be quantified according to characteristics such as voltage, current, electric field strength, magnetic field strength, frequency, power, or temperature. Absence of signal may refer to a signal level of zero or a signal level that is not meaningfully distinguished from noise.

As used herein, “crosstalk” refers to overlap of dye emission spectra. For example, crosstalk happens when one wants to collect green emission data, but the tail of blue emission overlaps green emission.

### Optical Detection Systems

In certain system embodiments, the optical signals may be directed through an optical train having a plurality of optical components. The optical signals may be directed to a detector (e.g., image sensor). In particular embodiments, the optical components of the optical train may be selectively moveable. As used herein, when the term “selectively” is used in conjunction with “moving” and similar terms, the phrase means that the position of the optical component may be changed in a desired manner. For example, at least one of the locations and the orientation of the optical component may be changed. The phrase “selectively moving” includes removing the optical component from the optical path, adjusting an orientation of the optical component in the optical path (e.g., rotating the optical component), or moving the optical component such that the orientation does not change, but the location of the optical component does change. In particular embodiments, the optical components may be selectively moved between imaging sessions. However, in other embodiments, the optical components may be selectively moved during an imaging session. As used herein, the term “xy coordinates” is intended to mean information that specifies location, size, shape, and/or orientation in an xy plane. The information may be, for example, numerical coordinates in a Cartesian system. The coordinates may be provided relative to one or both of the x and y axes or can be provided relative to another location in the xy plane. For example, coordinates of an analyte of an object may specify the location of the analyte relative to location of a fiducial or other analyte of the object.

As used herein, the term “z coordinate” is intended to mean information that specifies the location of a point, line or area along an axis that is orthogonal to an xy plane. In particular implementations, the z axis may be orthogonal to an area of an object that is observed by a detector. For example, the direction of focus for an optical system may be specified along the z axis.

As used herein, the term “fiducial” is intended to mean a distinguishable point of reference in or on an object. The point of reference may be, for example, a mark, second object, shape, edge, area, irregularity, channel, pit, post, or the like. The point of reference may be present in an image of the object or in another data set derived from detecting the object. The point of reference may be specified by an x and/or y coordinate in a plane of the object. Alternatively, or additionally, the point of reference may be specified by a z coordinate that is orthogonal to the xy plane, for example, being defined by the relative locations of the object and a detector. One or more coordinates for a point of reference may be specified relative to one or more other analytes of an object or of an image or other data set derived from the object.

In some implementations, acquired signal data may be transformed using an affine transformation. In some such implementations, template generation may make use of the fact that the affine transforms between color channels are consistent between runs. Because of this consistency, a set of default offsets may be used when determining the coordinates of the analytes in a specimen. For example, a default offsets file may contain the relative transformation (shift, scale, skew) for the different channels relative to one channel, such as the A channel. In other implementations, however, the offsets between color channels may drift during a run and/or between runs, making offset-driven template generation difficult. In such implementations, the methods and systems provided herein may utilize offset-less template generation, which is described further below.

Various types of fluorescence microscopy may be used with system embodiments described herein. Fluorescence microscopy may be performed using an optical detection system that includes a light source (e.g., lasers, light emitting diodes (LEDs)) tuned to wavelengths of light that induce excitation in the fluorescent dyes used for labelling a sample biological material or probe; one or more optical instruments, such as cameras, lenses, sensors, to capture signals emitted through induced excitation, and one or more processors for developing composite images from captured signals emitted from labelled targets within the optical elements' field of view (tile) in a given sequencing assay. For example, embodiments may be configured to perform at least one of conventional fluorescent imaging, epifluorescence imaging, total-internal-reflectance-fluorescence (TIRF) imaging, a time-delay integration (TDI) imaging (CCD-TDI or CMOS-TDI), or Super Resolution imaging, e.g., Structured Illumination Microscopy (SIM). Furthermore, the imaging sessions may include line scanning one or more samples such that a linear focal region of light is scanned across the sample(s). Imaging sessions may also include moving a point focal region of light in a raster pattern across the sample(s). Alternatively, one or more regions of the sample(s) may be illuminated at one time in a step and shoot manner.

In some embodiments, an optical detection system may include high-resolution optical components. Generally, an optical detection system may be limited by the optical resolution of the data capable of being detected by the optical components of the system. In microscopy, optical resolution is the shortest distance between two separate points in a microscope's field of view that can still be distinguished as distinct entities, i.e., the Rayleigh limit. For example, the optical resolution of such objects may be expressed as a function of a wavelength (λ) of light in the optical sequencing system, in which shorter wavelengths yield higher resolution, and an objective, or optical element (e.g., lens or lenses) used to gather the light from the target objects, which may be measured by a numerical aperture (NA). NA of an objective lens may be given by the formula nsine θ. where n is the index of refraction of the medium in which the lens is working (nair≈1), and θ is the half-angle of the maximum cone of light that can enter or exit the lens.

In one example, high-resolution images may be obtained through an optical detection system employing a high NA objective lens. An optical sequencing system using an objective lens having a relatively high NA is capable of resolving more closely adjacent point sources compared to a system characterized by a relatively lower NA. NA thus may determine the resolving power of an objective lens of an optical sequencing system. The higher the NA of the total system, the better the resolution. Higher quality detection lenses and other detection optical elements thus may be used to improve the optical resolution of the optical sequencing systems.

In another example, high-resolution images may be obtained through implementation of a subpixel imaging system, e.g., TDI using CCD- or CMOS-based sensors. Subpixel imaging is based on increasing a sample rate to raise the Nyquist frequency, which limits the highest frequency the optical sequencing system can reliably measure (e.g., translate digitally) to one half the sample rate at which the equipment operates. Subpixel imaging may be performed by staggering TDI sensors by a subpixel offset and subsampling a given collection area, which effectively doubles the Nyquist frequency along the offset-axis.

In yet another example, high-resolution images may be obtained through SIM or other SR techniques in connection, e.g., with optimized diffraction-limited imaging. SIM may be implemented by an optical sequencing system to take multiple images of a target object, with varying angles and phase displacements of structured illumination to generate a computational transform (Fourier transform) that is then used to reconstruct closely spaced, otherwise unresolvably high spatial frequency features, into lower frequency signals that may be sensed by an optical system without violating the Abbe diffraction limit. In that manner, captured raw images (e.g., six or nine images) of a same point source or point sources within a same tile may be assembled into a single image having an extended spatial frequency bandwidth, which may be transformed into real space to generate an image having a higher resolution than one captured by other imaging systems. Other apt SR microscopy systems include, e.g., direct stochastic optical reconstruction microscopy (dSTORM)); photo-activated localization microscopy (PALM)) and stimulated emission depletion microscopy (STED).

Various assay protocols involve performing a large number of controlled reactions on local support surfaces or within predefined reaction chambers. The occurrence of desired reactions may then be sensed or detected, and subsequent analysis may help identify or reveal properties of the kinetics and chemical(s) involved in the reaction. Many such protocols involve signal processing using fluorescence-based assays, which are among the most widely applied sensing techniques due to their high sensitivity, specificity, efficient operation, and the availability of diverse types of fluorophores that absorb and emit light covering a broad spectrum of wavelengths from ultraviolet to infrared.

The term “biological material” herein refers to a sample, typically derived from a biological fluid, cell, tissue, organ, or organism containing a nucleic acid or a mixture of nucleic acids containing at least one nucleic acid sequence that is to be sequenced and/or phased. Such samples include, but are not limited to sputum/oral fluid, amniotic fluid, blood, a blood fraction, fine needle biopsy samples (e.g., surgical biopsy, fine needle biopsy, etc.), urine, peritoneal fluid, pleural fluid, tissue explant, organ culture and any other tissue or cell preparation, or fraction or derivative thereof or isolated therefrom. Although the material is often taken from a human subject (e.g., patient), materials may be taken from any organism having chromosomes, including, but not limited to dogs, cats, horses, goats, sheep, cattle, pigs, etc. The material may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc.

The nanowell technology presently disclosed may be utilized for the optical detection, characterization, and/or identification of a variety of biological materials or other targeted analytes including, but are not limited to, nucleic acid materials (e.g., DNA, RNA, or analogs thereof), peptides, proteins, polysaccharides, cells, antibodies, epitopes, receptors, ligands, enzymes (e.g., kinases, phosphatases, or polymerases), small molecule drug candidates, cells, viruses, organisms, and the like.

A biological material herein may include nucleic acid materials such as target analytes, primers, templates, or probes. Nucleic acid materials may be referred to herein as “nucleic acids,” “nucleic acid molecules,” “nucleic acid materials,” “nucleic acid sequences,” “polynucleotides,” or “oligonucleotides,” and can comprise a polymeric form of nucleotides of any length, can comprise DNA and/or RNA, and can be single-stranded, double-stranded, or multiple stranded. One strand of a nucleic acid also refers to its complement. Nucleic acid analytes may be gDNA, including DNA variants (e.g., alleles, polymorphs, missense), mtDNA, mRNA, cDNA transcribed from mRNA, non-coding RNA, and small RNA. Nucleic acid materials herein may also include polynucleotide analogues, amplicons, conjugates, and substitutions, crosslinked polynucleotides, polynucleotide complexes, and non-natural polynucleotides, including, but not limited to, dideoxynucleotides, or biotinylated, aminated, deaminated, alkylated, benzylated, flourophor-labeled polynucleotides.

Nucleic acids in certain implementations may include, for instance, linear polymers of deoxyribonucleotides in 3′-5′ phosphodiester or other linkages, such as DNA, for example, single- and double-stranded DNA, genomic DNA, copy DNA or complementary DNA (cDNA), recombinant DNA, or any form of synthetic or modified DNA. In other implementations, nucleic acids include for instance, linear polymers of ribonucleotides in 3′-5′ phosphodiester or other linkages such as ribonucleic acids (RNA), for example, single- and double-stranded RNA, messenger (mRNA), copy RNA or complementary RNA (cRNA), alternatively spliced mRNA, ribosomal RNA, small nucleolar RNA (snoRNA), microRNAs (miRNA), small interfering RNAs (sRNA), piwi RNAs (piRNA), or any form of synthetic or modified RNA. Nucleic acids used in the compositions and methods of the present invention may vary in length and may be intact or full-length molecules or fragments or smaller parts of larger nucleic acid molecules. In particular implementations, a nucleic acid may have one or more detectable labels, as described elsewhere herein.

In various implementations, nucleic acids may be used as templates as provided herein (e.g., a nucleic acid template, or a nucleic acid complement that is complementary to a nucleic acid nucleic acid template) for particular types of nucleic acid analysis, including but not limited to nucleic acid amplification, nucleic acid expression analysis, and/or nucleic acid sequence determination or suitable combinations thereof.

In some implementations, the nucleic acid may comprise a plurality of copies of template nucleic acid and/or complements thereof, attached via their 5′ termini to the solid support. Such nucleic acid materials may be referred to “clusters” “colonies,” or “clonal populations.” The copies of nucleic acid strands making up the nucleic acid clusters may be in a single or double stranded form. Copies of a nucleic acid template that are present in a cluster can have nucleotides at corresponding positions that differ from each other, for example, due to presence of a label moiety. The corresponding positions can also contain analog structures having different chemical structure but similar Watson-Crick base-pairing properties, such as is the case for uracil and thymine. Nucleic acid clusters can optionally be created on solid supports by amplification, including, e.g., bridge amplification or exclusion amplification (ExAmp) techniques. Multiple repeats of a target sequence can be present in a single nucleic acid molecule, such as a concatemer created using a rolling circle amplification procedure. Such clusters may be characterized by a degree or ratio of monoclonality, or polyclonality.

A biological material herein may also include polypeptides, e.g., as analytes or reagent enzymes. Polypeptide analytes may include functional polypeptides acting, e.g., as effectors, inhibitors, modulators, mediators, transporters, or stimulators in connection with a specific activity affected by a target molecule. Reagent enzymes may include polypeptides involved in nucleic acid synthesis, extension, fragmentation, amplification, or ligation.

As used herein, the term “analyte” is intended to mean a point or area in a pattern that can be distinguished from other points or areas according to relative location. An individual analyte can include one or more sample cells, cellular constituents, or molecules of a particular type. For example, an analyte can include a single target nucleic acid molecule having a particular sequence or an analyte can include several nucleic acid molecules having the same sequence (and/or complementary sequence, thereof). Different molecules that are at different analytes of a pattern can be differentiated from each other according to the locations of the analytes in the pattern. Example analytes include without limitation, cavities or wells in a substrate, beads (or other particles) in or on a substrate, projections from a substrate, ridges on a substrate, pads of gel material on a substrate, or channels in a substrate.

As used herein, the term “fluorescent species” or “fluorescing species” refers to any target analyte, analyte conjugate, or other moiety that can be detected based on an optical field response to excitation. Fluorescing species may include a target analyte with inherent fluorescence (e.g., peptide tracers) or detectible label moieties. Exemplary labels for use consistent with various embodiments, for example, include chromophores; luminophores; fluorophores; optically encoded nanoparticles; particles encoded with a diffraction-grating; electrochemiluminescent labels such as Ru(bpy).sup.32+; or other species capable of detection based on an optical characteristic. In the instant disclosure the conjugated polymer fluorophores described herein are useful as a fluorescent species. Other fluorophores that may be useful include, for example, fluorescent lanthanide complexes, including those of Europium and Terbium, fluorescein, rhodamine, tetramethylrhodamine, eosin, erythrosin, coumarin, methyl-coumarins, pyrene, Malacite green, Cy3, Cy5, stilbene, Lucifer Yellow, Cascade Blue™, Texas Red, alexa dyes, phycoerythin, bodipy, and others known in the art.

The distances between analytes may be described in any number of ways. In some implementations, the distances between analytes may be described from the center of one analyte to the center of another analyte. In other implementations, the distances may be described from the edge of one analyte to the edge of another analyte, or between the outer-most identifiable points of each analyte. The edge of an analyte may be described as the theoretical or actual physical boundary on a chip, or some point inside the boundary of the analyte. In other implementations, the distances may be described in relation to a fixed point on the specimen or in the image of the specimen.

The size of an analyte on an array (or other object used in a method or system herein) may be selected to suit a particular application. For example, in some implementations, an analyte of an array may have a size that accommodates only a single nucleic acid molecule. A surface having a plurality of analytes in this size range is useful for constructing an array of molecules for detection at single molecule resolution. Analytes in this size range are also useful for use in arrays having analytes that each contain a colony of nucleic acid molecules. Thus, the analytes of an array may each have an area that is no larger than about 1 mm², no larger than about 500 μm², no larger than about 100 μm², no larger than about 10 μm², no larger than about 1 μm², no larger than about 500 nm², or no larger than about 100 nm², no larger than about 10 nm², no larger than about 5 nm², or no larger than about 1 nm². Alternatively, or additionally, the analytes of an array will be no smaller than about 1 mm², no smaller than about 500 μm², no smaller than about 100 μm², no smaller than about 10 μm², no smaller than about 1 μm², no smaller than about 500 nm², no smaller than about 100 nm², no smaller than about 10 nm², no smaller than about 5 nm², or no smaller than about 1 nm². Indeed, an analyte may have a size that is in a range between an upper and lower limit selected from those exemplified above. Although several size ranges for analytes of a surface have been exemplified with respect to nucleic acids and on the scale of nucleic acids, it will be understood that analytes in these size ranges may be used for applications that do not include nucleic acids. It will be further understood that the size of the analytes need not necessarily be confined to a scale used for nucleic acid applications.

For implementations that include an object having a plurality of analytes, such as an array of analytes, the analytes may be discrete, being separated with spaces between each other. An array useful in the context of the present technology may have analytes that are separated by edge-to-edge distance of at most 100 μm, 50 μm, 10 μm, 5 μm, 1 μm, 0.5 μm, or less. Alternatively, or additionally, an array may have analytes that are separated by an edge-to-edge distance of at least 0.5 μm, 1 μm, 5 μm, 10 μm.

The average pitch in a regular pattern may be at most 100 μm, 50 μm, 10 μm, 5 μm, 1 μm, 0.5 μm, or less. Alternatively, or additionally, the average pitch in a regular pattern may be at least 0.5 μm, 1 μm, 5 μm, 10 μm, 50 μm, 100 μm, or more. These ranges may apply to the maximum or minimum pitch for a regular pattern as well. For example, the maximum analyte pitch for a regular pattern may be at most 100 μm, 50 μm, 10 μm, 5 μm, 1 μm, 0.5 μm, or less; and/or the minimum analyte pitch in a regular pattern may be at least 0.5 μm, 1 μm, 5 μm, 10 μm, 50 μm, 100 μm, or more.

The density of analytes in an array may also be understood in terms of the number of analytes present per unit area. For example, the average density of analytes for an array may be at least about 1×10³ analytes/mm², 1×10⁴ analytes/mm², 1×10⁵ analytes/mm², 1×10⁶ analytes/mm², 1×10⁷ analytes/mm², 1×10⁸ analytes/mm², or 1×10⁹ analytes/mm², or higher. Alternatively, or additionally the average density of analytes for an array may be at most about 1×10⁹ analytes/mm², 1×10⁸ analytes/mm², 1×10⁷ analytes/mm², 1×10⁶ analytes/mm², 1×10⁵ analytes/mm², 1×10⁴ analytes/mm², or 1×10³ analytes/mm², or less.

The size and shape of analytes in a pattern may be determined by the size and shape of nanostructures in an array. For example, when observed in a two-dimensional plane, such as on the surface of an array, the analytes may appear rounded, circular, oval, rectangular, square, symmetric, asymmetric, triangular, polygonal, or the like. The analytes may be arranged in a regular repeating pattern including, for example, a hexagonal or rectilinear pattern. A pattern may be selected to achieve a desired level of packing. For example, round analytes are optimally packed in a hexagonal arrangement. Of course, other packing arrangements may also be used for round analytes and vice versa.

A pattern may be characterized in terms of the number of analytes that are present in a subset that forms the smallest geometric unit of the pattern. The subset may include, for example, at least about 2, 3, 4, 5, 6, 10 or more analytes. Depending upon the size and density of the analytes the geometric unit may occupy an area of less than 1 mm², 500 μm², 100 μm², 50 μm², 10 μm², 1 μm², 500 nm², 100 nm², 50 nm², 10 nm², or less. Alternatively, or additionally, the geometric unit may occupy an area of greater than 10 nm², 50 nm², 100 nm², 500 nm², 1 μm², 10 μm², 50 μm², 100 μm², 500 μm², 1 mm², or more. Characteristics of the analytes in a geometric unit, such as shape, size, pitch, and the like, may be selected from those set forth herein more generally with regard to analytes in an array or pattern.

An array having a regular pattern of analytes may be ordered with respect to the relative locations of the analytes but random with respect to one or more other characteristic of each analyte. For example, in the case of a nucleic acid array, the nuclei acid analytes may be ordered with respect to their relative locations but random with respect to one's knowledge of the sequence for the nucleic acid species present at any particular analyte. As a more specific example, nucleic acid arrays formed by seeding a repeating pattern of analytes with template nucleic acids and amplifying the template at each analyte to form copies of the template at the analyte (e.g., via cluster amplification, bridge amplification, or exclusion amplification (ExAmp)) will have a regular pattern of nucleic acid analytes but will be random with regard to the distribution of sequences of the nucleic acids across the array. Thus, detection of the presence of nucleic acid material generally on the array may yield a repeating pattern of analytes, whereas sequence specific detection may yield non-repeating distribution of signals across the array.

The technology described herein may be used in conjunction with a variety of nucleic acid sequencing techniques. Particularly applicable techniques are those in which nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are also applicable.

The term “sequence” in this context includes or represents a strand of nucleotides coupled to each other. The nucleotides may be based on DNA or RNA. It should be understood that one sequence may include multiple sub-sequences. For example, a single sequence (e.g., of a PCR amplicon) may have 350 nucleotides. The sample read may include multiple sub-sequences within these 350 nucleotides. For instance, the sample read may include first and second flanking subsequences having, for example, 20-50 nucleotides. The first and second flanking sub-sequences may be located on either side of a repetitive segment having a corresponding sub-sequence (e.g., 40-100 nucleotides). Each of the flanking sub-sequences may include (or include portions of) a primer sub-sequence (e.g., 10-30 nucleotides). For ease of reading, the term “sub-sequence” will be referred to as “sequence,” but it is understood that two sequences are not necessarily separate from each other on a common strand. To differentiate the various sequences described herein, the sequences may be given different labels (e.g., target sequence, primer sequence, flanking sequence, reference sequence, and the like). Other terms, such as “allele,” may be given different labels to differentiate between like objects.

The term “read” refers to a collection of sequence data that describes a fragment of a nucleotide sample or reference. The term “read” may refer to a sample read and/or a reference read. Typically, though not necessarily, a read represents a short sequence of contiguous base pairs in the sample or reference. The read may be represented symbolically by the base pair sequence (in ATCG) of the sample or reference fragment. It may be stored in a memory device and processed as appropriate to determine whether the read matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that may be used to identify a larger sequence or region, e.g., that may be aligned and specifically assigned to a chromosome or genomic region or gene.

In some embodiments, the process to determine the nucleotide sequence of a target nucleic acid may be an automated process using a sequencing-by-synthesis (“SBS”) technique. SBS techniques generally involve the enzymatic extension of a nascent nucleic acid strand through the iterative addition of nucleoside monomers against a template strand. SBS in general involves the enzymatic extension of a nascent copy strand through iterative addition and simultaneous detection of nucleoside monomers against a template strands. For each iteration (or cycle), labeled nucleoside monomers are detected through induced fluorescence as each monomer is added to the copy strand then replaced in a reverse-terminator process with non-labeled analogues before the start of a subsequent cycle. According to the example method, at a first cycle, (1) a buffer solution containing a mixture of four different labeled nucleoside derivatives (one for each of the four DNA nucleobases (A, G, C, T)) is provided to a cluster of template strands in the presence of polymerase; (2) a complementary nucleoside derivative is added to a nascent copy strand hybridized to each template strand via polymerase primer extension; (3) the added nucleoside derivatives are irradiated with incident light via an excitation source to induce fluorescence and emission of an output signal from the cluster; (4) the output signal is detected by one or more optical sensors through imaging of the signal as a point source at an addressed location on the optic substrate; and (5) information imparted in the imaged output signal is processed by a signal processor to record the nucleobase of the added nucleoside derivative and an address of the cluster on the optic substrate. Steps (1)-(5) are performed simultaneously for a plurality of clusters with a given frame (or tile) of the one or more optic sensors. Steps (1)-(5) are then repeated in subsequent cycles to n number of total cycles, where n is equal to the read length of the template strands in base pairs (bp).

Reads in the range of 50-100 bp may be obtained using an SBS-based technique paired with a single end sequencing chemistry, in which template strands are sequenced in one direction. Larger reads in the range of ˜300 to 800 bp may be obtained using an SBS-based technique with a paired-end sequencing chemistry to generate paired-end reads of each fragment in both forward and reverse directions. Thus, for example, continuous reads may be generated for 300 bp fragments using a 150 bp cycle kit, for 600 bp fragments using a 300 bp cycle kit, and so on. Still larger reads may be generated through computational leveraging. For example, reads may be generated for 800 bp fragments using a 300 bp cycle kit by inserting a known length between the paired ends (for simplicity, a 200 bp insert corresponding to the delta between the 800 bp fragment and the 2× 300 bp paired end reads) and inferring the sequence of the insert from the intersection of aligned read data in a pileup format. In one example, long insert paired-end reads are generated in combination with short insert paired reads sequenced at higher depth to infer long insert sequences.

Still larger reads in the range of several kilobases may be obtained using an SBS-based technique paired with mate pair sequencing chemistry such as Illumina Complete Long Reads (ICLR). Here, the sample gDNA may first be tagmented at desired fragment lengths with a Mate Pair Tagment Enzyme, which attaches a biotinylated junction adapter to each end of the tagmented molecule. The tagmented DNA molecules may then be circularized and the ends of the genomic fragment linked by the respective biotin junction adapters. Circularized molecules may then be re-fragmented yielding smaller fragments suitable for amplification and sequencing. Sub-fragments containing the original junction may then be enriched via the biotin tag in the junction adapter. After End Repair and A-tailing, TruSeq DNA adapters are then added, enabling amplification and sequencing. The short, fragmented reads may then be aligned to yield a long read for the tagmented fragment.

SBS may utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator may be effectively irreversible under the sequencing conditions used as is the case for traditional Sanger sequencing which utilizes dideoxynucleotides, or the terminator may be reversible.

Devices, assays, methods, and systems herein may also utilize probe-grafted arrays for screening biological molecules, such as nucleic acids and polypeptides, for a locus of interest. Such microarrays may include deoxyribonucleic acid (DNA) or ribonucleic acid (RNA) capture probes, which are specific for nucleotide sequences present in humans and other organisms. In certain applications, for example, individual DNA or RNA probes may be grafted at addressable reaction sites on an array surface. A test sample, such as from a known person or organism, can be exposed to the array, such that target nucleic acids hybridize to complementary probes grafted on the array. The probes can be labeled in a target specific process (e.g., due to labels present on the target nucleic acids or due to enzymatic labeling of the probes or targets that are present in hybridized form). The array may then be examined by scanning specific frequencies of light over the analytes to identify which target nucleic acids are present in the sample.

By way of example, a genotyping application as contemplated may be implemented to screen for the presence of a genetic locus of interest in a target nucleic acid sample. A locus of interest in a typical genotyping protocol, and as disclosed herein, may include, without limitation, polymorphs (e.g., single nucleotide polymorphs (SNPs), indels), short tandem repeats (STR), copy number variants (CNV), germline variants, methylation sites (e.g., CpG islands), and exogenous sequences (e.g., virus). Target nucleic acid samples herein may include polynucleotides of any length and may be derived from any number of genetic sources including from human or non-human organisms, and from individual organisms or organism populations. Samples herein may be obtained from a wide variety of genetic materials—e.g., gDNA, mtDNA, mRNA, cDNA transcribed from mRNA, non-coding RNA, and small RNA, polynucleotide conjugates, analogues, and amplicons.

Image-generating chip arrays provide a convenient format for assaying SNPs, particularly at commercial scale. An example workflow may begin with accession and extraction of a DNA sample, either from single cell source or a tissue sample. The extracted DNA sample may be amplified, usually off-chip in solution, and the amplicon output is then subjected to controlled enzymatic fragmentation. The processed DNA sample is loaded onto the image-generating chip and subjected to hybridization using locus specific oligo probes functionalized on the chip substrate. Allelic specificity of hybridized DNA is conferred by enzymatic base extension at 3′ end of the probe. Base extensions are applied fluorescent labels, imaged under excitation, and allele signal intensity data is used to perform genotype calling. An array may be functionalized with an individual probe or a population of probes. In the latter case, the population of probes at each analyte is typically homogenous having a single species of probe. For example, in the case of a nucleic acid array, each locus specific probe may be amplified to yield multiple nucleic acid molecules each having a common sequence. However, in some implementations the population of probes at a given reaction site of an array can be heterogeneous. Similarly, protein arrays can be functionalized with a single protein probe or a population of protein probes typically, but not always, having the same amino acid sequence. The probes can be attached to the surface of an array for example, via covalent linkage of the probes to the surface or via non-covalent interaction(s) of the probes with the surface.

In certain other implementations, the devices, methods, assays, and/or systems of the present disclosure may support a variety of fluorescence-based assays involving the detection and/or interaction of polypeptide materials, including, e.g., proteins, antibodies, epitopes, receptors, ligands, ligases, and enzymes (e.g., kinases, phosphatases, or polymerases). Polypeptide materials may include bioactive screening libraries containing collections of inhibitors, antagonists and agonists organized according to signaling pathways, including, e.g., DNA Damage/DNA Repair, Cell Cycle/Checkpoint, JAK/STAT Signaling Pathway, MAPK Signaling Pathway, GPCR/G protein, Angiogenesis, Immunology/Inflammation, ubiquitination, and proteolysis, among others.

In certain other implementations, nanostructured substrates of the present disclosure may support a variety of fluorescence-based high throughput screening (HTS) assays involving the detection, molecular interactions and/or activity of polypeptide materials, including, e.g., proteins, antibodies, epitopes, receptors, ligands, ligases, and enzymes (e.g., kinases, phosphatases, or polymerases). By way of example, a nanostructured substrate herein may support fluorescence polarization (FP)-based assay protocols, in which alterations in the apparent molecular weight of a fluorescent probe (or tracer) in solution are indicated by changes in the polarization of the sample's emitted light. for measuring ligand efficiency. The ability of FP assays to report on changes in molecular weight may be used as an investigative tool for variety of biological processes involving both molecular interactions (protein-protein, protein-peptide, protein-nucleic acid, protein-small molecule) and enzymatic activity (including substrate depletion and product formation). In one example, a nanostructured substrate in a microplate format may be used to support interrogation of candidate compounds selected, e.g., from a medicinal chemistry program a profile drug library for ligand efficiency using an FP-based assay. According to an example method, a ligase of interest with one or more defined ligand binding domains may be contacted with a known small-molecule peptide binder to form a binder-ligase complex formed via the one or more binding domains. The binder may be a tracer with inherent fluorescence, or the binder may be conjugated with a fluorescent species, e.g., a fluorophore. The complex solution is introduced to a nanostructured substrate constructed and arranged in accordance with any number of the examples provided herein such that the complex is immobilized across a nanowell array imprinted in the substrate. A baseline measure of fluorescence is first taken, and then a set of candidate compounds in solution is brought into contact with immobilized complexes across the array. The basis of the assay is that FP will increase when the fluorescent small molecule binder is bound to the protein. Equilibrium displacement of the protein-bound binder by a candidate compound will result in reduced FP, due to the faster rotation of the displaced binder, and no measurable change in fluorescence. If, on the other hand, the compound cannot bind the domain and the binder is not displaced, fluorescence will increase over time. The FP-based assay may be further utilized to quantitate substrate recruitment.

As another example, nanowell array technology herein may support fluorescence resonance energy transfer (FRET)-based assay protocols or Time-resolved FRET (TR-FRET) protocols to determine protein-protein interactions, e.g., ligand-receptor interaction. During FRET, a donor fluorophore excited by a light source may transfer its energy to a nearby acceptor fluorophore. The acceptor fluorophore absorbs the energy to produce a detectable light emission signal. This process results in the loss of fluorescence of the donor and the gain of fluorescence of the acceptor, both of which can be measured. Interactions between conjugated proteins of each respective fluorophore are determined based on FRET efficiency (E) which is based on donor and acceptor fluorophore proximity and is given by the formula E=R₀⁶/(R₀⁶+r⁶), where R₀ is the Förster radius, and r is the actual distance between the two fluorophores. The Förster radius is the distance at which 50% of the excitation energy is transferred from the donor to the acceptor, and the R₀ value usually lies between 10-100 Å (1-10 nm). FRET pairs with an R₀ value towards the higher end of this range are often preferred due to the increased likelihood of FRET occurrence. By way of example, FRET-based assays contemplated herein may be used to investigate enzyme-mediated ubiquitination pathways, inhibitors of protein:protein complex formation, protein dysregulation, and the like. FRET-based detection methods may also be used to sequence nucleic acid materials, e.g., detection of FRET interactions between a fluorophore-bearing polymerase and γ-phosphate-labeled nucleotides during SBS.

Similar to FRET, TR-FRET is based on the proximity of a donor label and an acceptor label, which have been brought together by a specific binding reaction. However, TR-FRET utilizes fluorescent lanthanide chelates (e.g., europium chelate, terbium chelate, fluorescein, europium cryptate) to avoid interference caused by short-lived emission from acceptor molecules excited directly, rather than by energy transfer.

Other applicable methodologies include, e.g., bimolecular fluorescence complementation (BiFC), green fluorescent protein GFP fluorescence, fluorescence activated cell sorting (FACS) and fluorescence intensity (FLINT)/Fluorescence Intensity Ratio (FIR).

As used herein, the term “optic solid support” or simply “solid support” refers to a structure upon which various chemistry (e.g., polymeric hydrogel, primers, etc.) may be added in connection with molecular analysis contemplated herein. The substrate may be a wafer, a panel, a rectangular sheet, a die, or any other suitable configuration. The substrate may generally be rigid and insoluble in an aqueous liquid. The substrate may be a single layer structure, or a multi-layered structure (e.g., including a support and a patterned material on the support). Examples of suitable substrates will be described further herein.

Various example embodiments described herein include methods and compositions for flow-cell based sequencing, e.g., of clonal populations of a nucleic acid library clustered on an array of solid support structures, orthogonal reagents, and complimentary chemistry for functionalization of a flow cell surface for selective capture, in situ enrichment, imaging, and traceless release of a nucleic acid sample in a sequencing cycle. As used herein, a flow cell is a vessel having a flow channel that is in fluid communication with at least one unmodified surface or at least one surface modified with a first member of a transition metal complex binding pair. The unmodified or modified surface is capable of attaching surface chemistry that to be used in during a nucleic acid analysis and is capable of releasing the surface chemistry either electrochemically or upon exposure to visible light. The flow cell may also include an inlet for delivering reagent(s) to the flow channel and an outlet for removing reagent(s) from the flow channel. The flow cell enables the detection of the reactions involving the surface chemistry. For example, the flow cell may include one or more transparent surfaces, which allow for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a patterned or nonpatterned structure and a lid. In other examples, the flow channel may be defined between two patterned or non-patterned structures that are bonded together.

Any of a variety of array configurations (also referred to as “microarrays” or “microarray chips”) known in the art can be used in a system, method or device set forth herein, including, e.g., with assay workflows for SNP genotyping, epigenetics, genotyping, biomarker profiling, translation profiling, pathway identification mutations, allele specific primer extension (APSE), and gene expression profiling.

By way of example, nanostructured substrates herein may be implemented on microarrays useful, e.g., in connection with genotyping assays, systems and platforms. Microarrays typically include deoxyribonucleic acid (DNA) or ribonucleic acid (RNA) probes. These are specific for nucleotide sequences present in humans and other organisms. In certain applications, for example, individual DNA or RNA probes can be attached to individual analytes of an array. A test sample, such as from a known person or organism, can be exposed to the array, such that target nucleic acids (e.g., gene fragments, mRNA, or amplicons thereof) hybridize to complementary probes at respective analytes in the array. The probes can be labeled in a target specific process (e.g., due to labels present on the target nucleic acids or due to enzymatic labeling of the probes or targets that are present in hybridized form at the analytes). The array can then be examined by scanning specific frequencies of light over the analytes to identify which target nucleic acids are present in the sample.

Microarrays may also be used for genetic sequencing and similar applications. In general, genetic sequencing comprises determining the order of nucleotides in a length of target nucleic acid, such as a fragment of DNA or RNA. Relatively short sequences are typically sequenced at each analyte, and the resulting sequence information may be used in various bioinformatics methods to logically fit the sequence fragments together so as to reliably determine the sequence of much more extensive lengths of genetic material from which the fragments were derived. Automated, computer-based algorithms for characteristic fragments have been developed, and have been used more recently in genome mapping, identification of genes and their function, and so forth. Microarrays are particularly useful for characterizing genomic content because a large number of variants are present, and this supplants the alternative of performing many experiments on individual probes and targets. The microarray is an ideal format for performing such investigations in a practical manner.

The term “threshold” herein refers to a numeric or non-numeric value that is used as a cutoff to characterize a sample, a nucleic acid, or portion thereof (e.g., a read). A threshold may be varied based upon empirical analysis. The threshold may be compared to a measured or calculated value to determine whether the source giving rise to such value suggests should be classified in a particular manner. Threshold values can be identified empirically or analytically. The choice of a threshold is dependent on the level of confidence that the user wishes to have to make the classification. The threshold may be chosen for a particular purpose (e.g., to balance sensitivity and selectivity). As used herein, the term “threshold” indicates a point at which a course of analysis may be changed and/or a point at which an action may be triggered. A threshold is not required to be a predetermined number. Instead, the threshold may be, for instance, a function that is based on a plurality of factors. The threshold may be adaptive to the circumstances. Moreover, a threshold may indicate an upper limit, a lower limit, or a range between limits. As an example, threshold reliability of sequencing (or base call) data generated in a nucleic acid sequencing operation may be based on a percent passing filter (% PF), which may be calculated based on the computational application of a chastity filter to a point source (e.g., a cluster), where chastity is defined as the ratio of the brightest base intensity divided by the sum of the brightest and second brightest base intensities. A point source “passes filter” if no more than a defined number of base calls has a chastity value below a set threshold.

In some implementations, a metric or score that is assigned to sequencing data may be compared to the threshold. As used herein, the terms “metric” or “score” may include values or results that were determined from the sequencing data or may include functions that are based on the values or results that were determined from the sequencing data. Like a threshold, the metric or score may be adaptive to the circumstances. For instance, the metric or score may be a normalized value. As an example of a score or metric, the quality of a base call determination in a nucleic acid sequencing protocol may be reflected in a “Q-score” based on a PHRED-scaled probability ranging from 0-50 inversely proportional to the probability that a single sequenced base is correct. For example, a thymine (T) base call with Q of 20 is considered likely correct with a probability of 99.99%. Any base call with Q<20 may be considered low quality.

Optical detection systems herein may perform computation-based image or spectral data analysis using one or more automated subsystems. Subsystems may include a processor; a storage capacity; and a program(s) for image analysis, the program comprising instructions for processing a first data set for storage and the second data set for analysis, wherein the processing comprises acquiring and/or storing the first data set on the storage device and analyzing the second data set when the processor is not acquiring the first data set. In certain aspects, the program includes instructions for identifying at least one instance of a conflict between acquiring and/or storing the first data set and analyzing the second data set; and resolving the conflict in favor of acquiring and/or storing image data such that acquiring and/or storing the first data set is given priority. In certain aspects, the first data set comprises image files obtained from an optical imaging device. In certain aspects, the system further comprises an optical imaging device. In some aspects, the optical imaging device comprises a light source and a detection device.

Generally, several implementations will be described herein with respect to methods of analysis. It will be understood that systems are also provided for carrying out the methods in an automated or semi-automated way. Accordingly, this disclosure provides computation-based template generation and base calling systems, wherein the systems can include a processor; a storage device; and a program for image analysis, the program including instructions for carrying out one or more of the methods set forth herein. Accordingly, the methods set forth herein can be carried out on a computer, for example, performance of real time analysis of image and sequence data generated during a DNA sequencing operation.

Also provided herein are systems for performing secondary and/or tertiary analysis of molecular analysis performed using devices, methods, assays, and/or systems presently described. For example, computational systems implementing an SV calling pipeline for generation of diploid assemblies and SV call sets from whole genome or whole exome sequencing data using sequencing devices, methods, assays, and systems described herein. Tertiary analysis applications may include comprehensive genomic profiling, Genome-wide Association Studies (GWAS), Variant to Function (V2F), QTL mapping, missense studies, loss of function analysis, gain of function analysis, conservation, depletion, deletion analyses.

The technology disclosed may use neural networks to improve the quality and quantity of nucleic acid sequence information that can be obtained from a nucleic acid sample such as a nucleic acid template or its complement, for instance, a DNA or RNA polynucleotide or other nucleic acid sample. Accordingly, certain implementations of the technology disclosed provide higher throughput polynucleotide sequencing, for instance, higher rates of collection of DNA or RNA sequence data, greater efficiency in sequence data collection, and/or lower costs of obtaining such sequence data, relative to previously available methodologies.

The technology disclosed may utilize neural networks to identify the center of a nucleic acid clusters described herein and to analyze optical signals that are generated during sequencing of such clusters, to discriminate unambiguously between adjacent, abutting or overlapping clusters in order to assign a sequencing signal to a single, discrete source cluster. These and related implementations thus permit retrieval of meaningful information, such as sequence data, from regions of high-density cluster arrays where useful information could not previously be obtained from such regions due to confounding effects of overlapping or very closely spaced adjacent clusters, including the effects of overlapping signals (e.g., as used in nucleic acid sequencing) emanating therefrom.

A number of tasks of automated systems described herein may be offloaded from software to configurable hardware, e.g., FGPAs, for acceleration of computational processing. For example, one or more FPGAs may be configured to perform tasks at various stages of secondary analyses performed herein. For instance, FPGA may be parallelized for multithreading, including, e.g., multithreading variant calling by chromosome. FPGAs may be instantiated with graphs or patterns for do novo assembly. And they may be configured for highly repeated processes, such as hidden Markov Models or Smith-Waterman algorithms.

The illustrated embodiments of the Figures herein are disclosed in the context assay protocols to which the nanowell array technology of the present disclosure has application. These include DNA sequencing using sequencing-by-synthesis (SBS) techniques on NGS platforms; screening nucleic acid materials for loci of interest using microarray formats, including, e.g., single nucleotide polymorph (SNP) screens; and high throughput screening (HTS) for protein: protein interaction investigation. These protocols, while well suited to the devices, methods, assays, and systems of the present disclosure, serve merely to highlight the utility and various advantages for particular embodiments herein. The technology presently disclosed, including as disclosed through the illustrated embodiments, may be utilized in connection with any optics-based assay, including fluorescence-based assay involving the processing of optic information imparted in signals emitted by target analytes under fluorescence and captured by a sensor at different time points, spatial locations, or other temporal or physical perspectives as images and/or spectral data.

### Example Supported Flow Cell Architecture

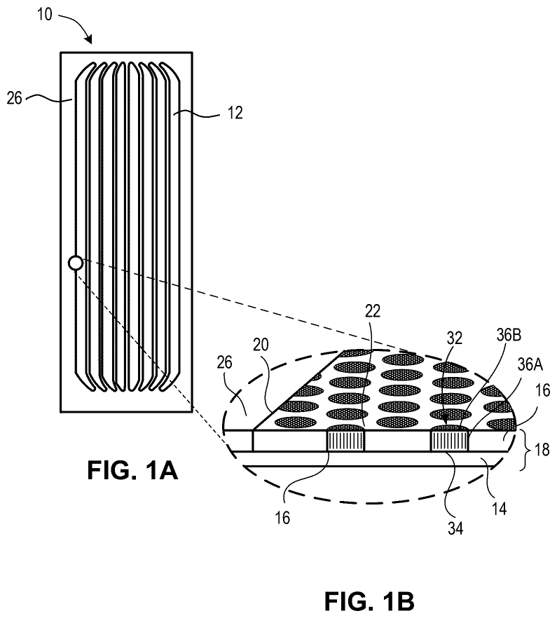

One example of supported flow cell architecture is the flow cell 10 as shown in FIGS. 1A and 1B. Generally, flow cell 10 may include a patterned structure, e.g., an array of depressions 32, as shown in FIG. 1B, and the patterned structure may be organized into lanes, each separated by non-patterned, non-functionalized interstitial regions, which may be bonded to a lid 20 to form flow channels 12 along each lane of patterned structure. The example shown in FIG. 1A includes eight flow channels 12. While eight flow channels 12 are shown, it is to be understood that any number of flow channels 12 may be included in the flow cell 10 (e.g., a single flow channel 12, four flow channels 12, etc.). Each flow channel 12 may be isolated from another flow channel 12 so that fluid introduced into a flow channel 12 does not flow into adjacent flow channel(s) 12. Some examples of the fluids introduced into the flow channel 12 may introduce sample and reaction components (for NGS: e.g., DNA sample, polymerases, sequencing primers, nucleotides, etc.), washing solutions, deblocking agents, etc.

As illustrated in FIG. 1B, the flow channel 12 may include a multi-layered or composite structure 18, which includes, a minimum, a single layer base support 14 overlayed with a resin or other appropriate film layer 16.

The support layer may be any suitable low-background material, including materials exhibiting both high transmissivity and high fluorescence transparency, particularly for use as solid supports for fluorescence-based imaging implementations. Examples of suitable single layer base supports 14 include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like. In one example, single layer base supports 14 is a glass, for example, alkaline earth boro-aluminosilicate glass (e.g., EAGLE XG® (Corning, NY)), which has an annealing point (10¹³ poises) rated ˜1332° F.

The resin film layer 16 may have thickness sufficient to accommodate the depth of depression 32, which can range, for example, between 0.01 nm to 450 nm depending on the application. As discussed in greater detail here, in one example (shown in FIG. 1B), the thickness of the resin film layer may be coterminous with the depth of depression 32, such that a base portion of the inner surface of depression 32 exposes a surface of the single layer base supports 14. Alternatively, the thickness of the resin film layer may accommodate the entire depth of depression 32, such the entire inner surface of depression 32 is formed in the material of the resin film layer.

The layout or pattern may be characterized with respect to the density (number), porosity (pore area as a % portion of film area), pitch (center-point distance between neighboring wells of depressions 32 in nm), diameter (cell and/or well of depressions 32 in nm), and well depth (nm) of the depressions 32 in a defined area.

For example, the depressions 32 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the depressions 32 separated by less than about 100 nm, a medium density array may be characterized as having the depressions 32 separated by about 400 nm to about 1 μm, and a low-density array may be characterized as having the depressions 32 separated by greater than about 1 μm.

The layout or pattern of the depressions 32 may also or alternatively be characterized in terms of the average pitch, or the spacing from the center of one depression 32 to the center of an adjacent depression 32 (center-to-center spacing) or from the right edge of one depression 32 to the left edge of an adjacent depression 32 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern can be between one of the lower values and one of the upper values selected from the ranges above. In some embodiments, the depressions 32 are nanowells and have an average pitch (center-to-center spacing) about 250 nm or greater, 300 nm or greater, 350 nm or greater, 400 nm or greater, 450 nm or greater, 500 nm or greater, 550 nm or greater, 600 nm or greater, 650 nm or greater, or 700 nm or greater, or may be in a range between about 250 nm and 800 nm, 300 nm and 750 nm, 350 nm and 700 nm, 400 nm and 650 nm, 450 nm and 600 nm, 500 nm and 550 nm. In an example, the depressions 32 are nanowells and have an average pitch (center-to-center spacing) between about 350 nm and 750 nm. While example average pitch values have been provided, it is to be understood that other average pitch values may be used.

The depressions 32 may be characterized by the geometric shape of a cross-section of the depression 32 taken parallel to a predetermined plane, such as a face of interstitial regions 22, or by the volume, opening area, depth, diameter, length, or width of the depression 32, or by a combination thereof. For example, the depressions 32 may be nanowells with a natural hexagonal morphology, or the depressions 32 may be nanowells with hexagonal openings, or the depressions 32 may be nanowells reconfigured as cylindrical structures with substantially circular openings. For another example, the opening area can range from about 1×10⁻³ μm² to about 100 μm², e.g., about 1×10⁻² μm², about 0.1 μm², about 1 μm², at least about 10 μm², or more, or less. In another example, the volume can range from about 1×10⁻³ μm³ to about 100 μm³, e.g., about 1×10⁻² μm³, about 0.1 μm³, about 1 μm³, about 10 μm³, or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less. In another example, the depressions 32 are nanowells and the average depth is 150 nm or greater, 200 nm or greater, 250 nm or greater, 300 nm or greater, 350 nm or greater, or 400 nm or greater, or may be in a range between about 150 nm and 500 nm, 200 nm and 450 nm, or 250 nm and 400 nm., or 300 nm and 350 nm.

The single layer base support 14 (whether used singly or as part of the multi-layered structure 18) may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a single base support 14 with any suitable dimensions may be used.

In an example, the flow channel 12 has a rectangular configuration. The length and width of the flow channel 12 may be selected so a portion of the single base support 14 or an outermost layer of the multi-layered structure 18 surrounds the flow channel 12 and is available for attachment to a lid (not shown) or another patterned or non-patterned structure. The surrounding portions are the bonding regions 26.

The depth of the flow channel 12 can be as small as a monolayer thick when microcontact, aerosol, or inkjet printing is used to deposit a separate material over the bonding region 26 that defines the flow channel 12 walls. In other examples, a thicker spacer layer may be applied to bonding region 26 so that the spacer layer defines at least a portion of the walls of the flow channel 12. As one example, the spacer layer can be a radiation-absorbing material that aids in bonding. In these examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than or between the values specified above.

FIG. 1B depicts an example architecture within the flow channel 12. The architecture shown in FIG. 1B is a patterned structure that includes depressions 32 and interstitial regions 22 defined in the resin film layer 16 of the multi-layer structure 18, or alternatively or in addition to, the single base support 14. In one example, the interstitial regions 22 are non-functionalized, and substantially planer and featureless.

For the patterned structure, many different layouts of the depressions 32 may be envisaged, including regular, repeating, and non-regular patterns. In an example, the depressions 32 are disposed in self-ordered hexagonal grid. As discussed in greater detail herein, other layouts may be engineered by pre-patterning processing using, for example, imprinting techniques, masking, photolithography, nanoimprint lithography (NIL), stamping techniques, embossing techniques, molding techniques, microetching techniques, or a combination thereof. for example, rectilinear (rectangular) layouts, triangular layouts, and so forth. In some examples, the layout or pattern can be an x-y lattice format in rows and columns. In other examples, the layout or pattern can be a repeating arrangement of depressions 32 and the interstitial regions 22. In still other examples, the layout or pattern can be a random arrangement of the depressions 32 within the interstitial regions 22.

Support materials for use with nanoimprint lithography (NIL) and other imprinting techniques may be selected from any suitable UV-curable resins, including tripropyleneglycol diacrylate (TPGDA resin), polypropyleneglycol diacrylate (PPGDA resin), poly-urethane acrylate (PUA resin), and fluoroacrylates, e.g., perfluoropolyether (PFPE)-urethane methacrylate (MD 700 resin).

The layout or pattern may be characterized with respect to the density (number) of the depressions 32 in a defined area. For example, the depressions 32 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high-density array may be characterized as having the depressions 32 separated by less than about 100 nm, a medium density array may be characterized as having the depressions 32 or functionalized pads 28 separated by about 400 nm to about 1 μm, and a low density array may be characterized as having the depressions 32 separated by greater than about 1 μm.

The layout or pattern of the depressions 32 may also or alternatively be characterized in terms of the average pitch, or the spacing from the center of one depression 32 to the center of an adjacent depression 32 (center-to-center spacing) or from the right edge of one depression 32 to the left edge of an adjacent depression 32 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of can be between one of the lower values and one of the upper values selected from the ranges above. In an example, the depressions 32 have a pitch (center-to-center spacing) of about 1.5 μm. While example average pitch values have been provided, it is to be understood that other average pitch values may be used.

The size of each depression 32 may be characterized by its volume, opening area, depth, and/or diameter or length and width. For example, the volume can range from about 1×10-3 μm3 to about 100 μm³, e.g., about 1×10-2 μm³, about 0.1 μm³, about 1 μm3, about 10 μm3, or more, or less. For another example, the opening area can range from about 1×10-3 μm² to about 100 μm2, e.g., about 1×10-2 μm2, about 0.1 μm2, about 1 μm2, at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

Each of the architectures also includes a solid support 34 for staging molecular analyses herein. The solid support 34 includes the polymeric hydrogel and primers 36A, 36B. In the patterned structure of FIG. 1B, the solid support 34 is located within the depression 32

Each of the architectures also includes the active area 32. The active area 32 may include a polymeric hydrogel and primers 36A, 36B. In the patterned structure of FIG. 1B, the active area 32 is located within the depression 32.

The polymeric hydrogel may be any gel material that can swell when liquid is taken up and can contract when liquid is removed, e.g., by drying. In an example, the polymeric hydrogel includes an acrylamide copolymer, such as poly(N-(5-azidoacetamidylpentyl) acrylamide-co-acrylamide, PAZAM or other forms of the acrylamide copolymer In some examples, PAZAM and other forms of the acrylamide copolymer are linear polymers. In some other examples, PAZAM and other forms of the acrylamide copolymer are lightly cross-linked polymers. In other examples, the gel material may be a variation of the structure (I). In one example, the acrylamide unit may be replaced with N,N-dimethylacrylamide.

It is to be understood that other polymeric hydrogels may be used, as long as they are functionalized to graft oligonucleotide primers 36A, 36B thereto. Some examples of suitable the polymeric hydrogel include functionalized polysilanes, such as norbornene silane, azido silane, alkyne functionalized silane, amine functionalized silane, maleimide silane, or any other polysilane having functional groups that can attach the desired primer set 36A, 36B. Other examples of suitable polymeric hydrogels 34 include those having a colloidal structure, such as agarose; or a polymer mesh structure, such as gelatin; or a cross-linked polymer structure, such as polyacrylamide polymers and copolymers, silane free acrylamide (SFA), or an azidolyzed version of SFA. Examples of suitable polyacrylamide polymers may be synthesized from acrylamide and an acrylic acid or an acrylic acid containing a vinyl group, or from monomers that form [2+2] photo-cycloaddition reactions. Still other examples of suitable polymeric hydrogels include mixed copolymers of acrylamides and acrylates. A variety of polymer architectures containing acrylic monomers (e.g., acrylamides, acrylates etc.) may be utilized in the examples disclosed herein, such as branched polymers, including dendrimers, and the like. For example, the monomers (e.g., acrylamide, etc.) may be incorporated, either randomly or in block, into the branches (arms) of a dendrimer.

The polymeric hydrogel may be formed using any suitable copolymerization process. The polymeric hydrogel may be deposited using any of the methods disclosed herein. For at least some of the deposition techniques, the polymeric hydrogel may be incorporated into a mixture, e.g., with water or with ethanol and water, and then applied. The attachment of the polymeric hydrogel to the underlying base support 14 or resin layer 16 of the multi-layer structure 18 may be through covalent bonding. In some instances, the underlying base support 14 or layer 16 may first be activated, e.g., through silanization or plasma ashing. As discussed in greater detail herein, with respect to certain embodiments herein, each of the architectures also includes the primer 36A, 36B attached to the polymeric hydrogel.

The flow cells may be used in a variety of sequencing approaches or technologies, including SBS. In SBS, extension of a nucleic acid primer (e.g., a sequencing primer) along a nucleic acid template (i.e., the sequencing template) is monitored to determine the sequence of nucleotides in the template. The underlying chemical process can be polymerization (e.g., catalyzed by a polymerase enzyme) or ligation (e.g., catalyzed by a ligase enzyme). In a particular polymerase-based SBS process, fluorescently labeled nucleotides are added to the sequencing primer (thereby extending the sequencing primer) in a template dependent fashion such that detection of the order and type of nucleotides added to the sequencing primer can be used to determine the sequence of the template.

Sequencing methodologies for nucleic acid samples on NGS platforms commonly deploy DNA libraries in which a DNA target (e.g., genomic DNA (gDNA), or complimentary DNA (cDNA)) is processed into fragments and ligated with technology-specific adaptors. Fragments may also be ligated with sample source-specific barcoding in multiplexed operations. NGS workflow using SBS involves loading a DNA library onto a flow cell and hybridizing individual DNA fragments to adapter-specific complimentary oligonucleotides (oligos) covalently bound to the flow cell surface.

Amplification may also be performed using an exclusion amplification (ExAmp) technique. ExAmp cluster generation is particularly appropriate for optimizing monoclonality of amplicon populations on a given reaction site. ExAmp chemistry carries out seeding and cluster amplification steps simultaneously such that amplification of a first seeding event on a given reaction site occurs nearly instantaneously and the amplification rate far exceeds the reaction rate for seeding. In that manner, the ExAmp amplification of a fragment seeded to a reaction site prevents further seeding by other fragments, thus reducing the occurrence of undesirable polyclonal clustering.

A sequencing primer may be introduced that hybridizes to a complementary sequence on the template strand. This sequencing primer renders the template polynucleotide strand ready for sequencing. The 3′-ends of the templates and any flow cell-bound primers (not attached to the copy) may be blocked to prevent interference with the sequencing reaction, and in particular, to prevent undesirable priming.

To initiate sequencing, an incorporation mix may be added to the flow cell. In one example, the incorporation mix may include a liquid carrier, fluorescently labeled nucleoside triphosphates (NTPs), and one or more replication enzymes (e.g., polymerase). The fluorescently labeled NTPs may include a 3′ OH blocking group. When the incorporation mix is introduced into the flow cell in a given cycle, the fluid enters a flow channel and flows into nanowell to contact the reaction sites hosting the clustered template strands.

In certain embodiments, an unlabeled nucleotide may be incorporated and then a fluorescent label may be added to the nucleoside (post-incorporation of the labeling). In this circumstance, an unlabeled nucleoside can comprise a functional group that can react with a fluorescent label to form the fluorescent nanoparticle.

Common click chemistries, known to those in the art, can be used in the post incorporation of fluorescent labels. Strong physical binding, such as avidin-biotin, may also be utilized. See, for example, Sweenk, et al., Current Opinion in Chemical Biology 2021, 60:79-88.

The fluorescently labeled nucleotides may be added to the sequencing primer one nucleotide per cycle in a template dependent fashion such that detection of the order and type of nucleotides added to the sequencing primer can be used to determine the sequence of the template. More particularly, one of the nucleotides is incorporated, by a respective polymerase, into a nascent strand that extends the sequencing primer and that is complementary to the template polynucleotide strand.

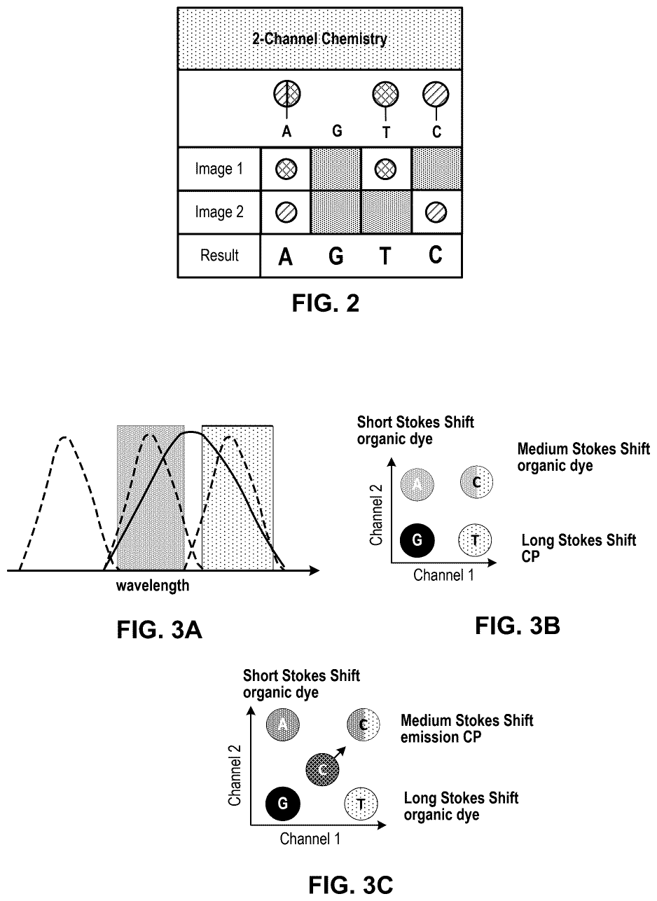

In certain sequencing protocols herein, e.g., SBS, imaging is performed at each of two wavelengths, e.g., blue and green, and decoded as shown in FIG. 2. This chemistry is a two excitation/two wavelength process. Labeled nucleotide bases (or clusters) seen in blue or green images (illustrated as diagonal lines and cross hatch, respectively) are interpreted as C and T bases, respectively. Bases observed in both blue and green images are flagged as A bases, while unlabeled clusters are identified as G bases. In certain aspects, the system comprises a flow cell that is configured to deliver additional labeled nucleotide bases to the array of molecules, thereby producing a plurality of cycles of color images. The instant disclosure builds on this technology by using one excitation/two channel processes. By reducing from two to one excitation, the instantly disclosed process allows for greater efficiency and lower cost sequencing.

Image data of nucleotides incorporated during a given cycle may be collected by an imager, e.g., in a fluorescence microscopy system using a point, area, or line imaging technique. Image data may be taken for a collection area, such as corresponding to a tile of a flow cell. Some implementations of line imaging correspond to discrete point-and-shoot operation. Point imaging collects point-images as relatively smaller collections of one or more pixels, collecting image data for larger areas by progressing left to right along an x axis of the flow cell surface, then up along a y axis, then again left to right, and so forth, via relative movement of the imager and the flow cell, such as in discrete point-and-shoot operation. Area imaging collects area-images as relatively larger collections of pixels, such as in a rectangular (e.g., square) shape. Area image collection progresses similarly to that of point imaging, by progressing from left to right, then up, then again left to right, and so forth, via relative movement of the imager and the flow cell, such as in discrete point-and-shoot operation. Line imaging collects line-images as collections of pixels corresponding to a rectangular region of a relatively high aspect ratio, such as a single pixel high and several pixels wide corresponding to the collection area width. Line image collection progresses a line at a time in a direction orthogonal to the line, via relative movement of the imager and flow cell. Some implementations of line imaging correspond to continuous scanning operation, e.g., the imager and the flow cell are in continuous movement with respect to each other and image capture is performed during the movement. Some implementations of continuous scanning operation are performed using Time Delay Integration (TDI).

In some examples, the nucleotides may further include a reversible termination property (e.g., 3′ OH blocking group) that terminates further primer extension once a nucleotide has been added to the sequencing primer. For example, a nucleotide analog having a reversible terminator moiety may be added to the sequencing primer such that subsequent extension cannot occur until a deblocking agent is delivered to remove the moiety. Thus, for examples that use reversible termination, a deblocking reagent can be delivered to the flow cell after detection occurs.

Wash(es) may take place between the various fluid delivery steps. The SBS cycle may then be repeated n times to extend the sequencing primer by n nucleotides, thereby detecting a sequence of length. In some examples, the forward strands may be sequenced and removed, and then reverse strands are constructed and sequenced as described herein.

Base call outputs generated from NGS and other sequencing methodologies may be output to a FASTA text file or binary counterpart. A FASTA file may include a text file that contains sequence data from clusters that pass filter on a flow cell. A FASTA file may also include corresponding quality scores of the sequence.

In one example, a method for optical detection of a nucleic acid sample in a sequencing protocol is provided. According to the example method, a DNA library may be loaded onto optic support layer of flow cell device, where constituent fragment strands are flowed across an array of nanowells impressed in the optic support layer and individual strands are absorbed by nanowells of the array at a substantially 1:1 basis, and each absorbed strand is immobilized at a reaction site of respective nanowell through interaction between the strand adaptor and a capture primer covalently bound to the surface of the reaction site. Each immobilized strand is then amplified (e.g., using bridge or exclusion amplification) to yield a substantially monoclonal cluster of template strands within each respective nanowell.

Each of the resulting clusters is then sequenced using, e.g., a sequencing-by-synthesis (SBS) technique. According to the example method, in a first cycle, (1) a buffer solution containing a mixture of four different labeled nucleoside derivatives (one for each of the four DNA nucleobases (A, G, C, T)) may be provided to a cluster of template strands in the presence of polymerase; (2) a complementary nucleoside derivative may be added to a nascent copy strand hybridized to each template strand via polymerase primer extension; (3) the added nucleoside derivatives may be irradiated with incident light via an excitation source to induce fluorescence and emission of an output signal from the cluster; (4) the output signal may be detected by one or more optical sensors through imaging of the signal as a point source at an addressed location on the optic substrate; and (5) information imparted in the imaged output signal may be processed by a signal processor to record the nucleobase of the added nucleoside derivative and an address of the cluster on the optic substrate. Steps (1)-(5) may be performed simultaneously for a plurality of clusters with a given frame (or tile) of the one or more optic sensors. Steps (1)-(5) may then be repeated in subsequent cycles to n number of total cycles, where n is equal to the size of the template strands in base pairs (bp).

With a one exposure (1×) two-channel system, the added nucleoside derivatives are irradiated with one excitation source to induce florescence in step (3) and the output signal is detected by two sensors in step (4). Using a nucleoside with a long Stokes shift fluorescence label in addition to a lower Stokes shift fluorescence label, allows for this type of operation. For example, as discussed above, a first type nucleobase may be labeled with a blue-blue fluorophore (long Stokes shift), a third-type nucleobase labeled with both the blue-blue fluorophore and the blue-green fluorophore or the third-type nucleobase can be labeled with a fluorophore having an emission between blue emission of the first-type nucleobase and the green emission of the second type nucleobase, and the fourth type nucleobase is unlabeled.

Photon budget requirements for sequencing methodologies on NGS platforms, particularly with SBS-based processes, are relatively high. In each cycle, a plurality of clusters is exposed to excitation power. The total number of cycles corresponds to the read length of bases on the template strands of each of the clusters. Example read lengths may be 50, 75, 150, and 300 base pairs, which correspond to a respective number of total cycles. Moreover, the fluorescence chemistry of NGS requires as many as four images per cycle to capture fluorescence of each of the four base types added in a given round. As just one example, a typical step and shoot sequencing operation using a flow cell format and 300 cycle SBS chemistry may obtain two images per sequencing cycle per tile (using two-channel chemistry), where the total number of cycles is equal to the length in bp of the DNA template strand, viz., 300 bp, and the total number of tiles image may be 130 or greater, resulting in a total number of imaging steps that can exceed 78,000 (130×300×2).

Advantageously, step and shoot sequencing operations using 1×, two channel chemistry enabled by fluorophores of the present disclosure can reduce the total number of imaging steps by half, e.g., 39,000, using a flow cell format and 300 cycle SBS chemistry.

### Conjugated Polymer (CP) Strategy

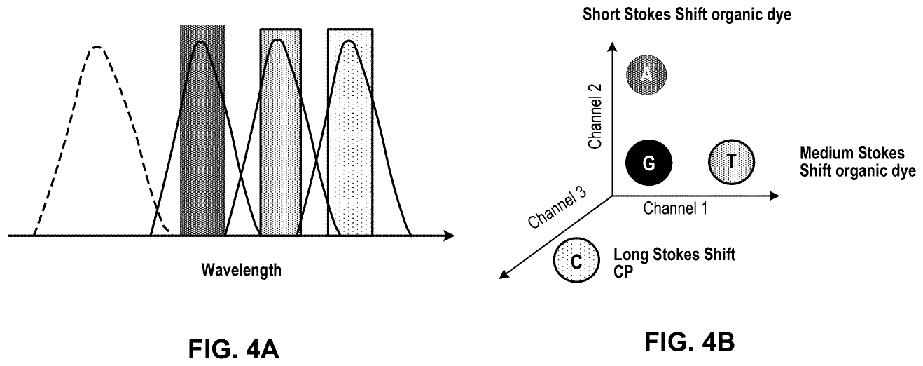

A proposed base labeling strategy for 1Ex-2Ch (1 excitation frequency/2 channel), 2Ex-3Ch (2 excitation frequences/3 channel) and 1Ex-3Ch (1 excitation frequency/3 channel) includes the use of conjugated polymers as fluorophores to allow more tunable optical properties, enhanced brightness and photostability. For example, in a typical 1Ex-2Ch detection scheme, CPs and organic dyes that share a common excitation range could be combined, with the CP acting as the long Stokes shift fluorophore (FIG. 3B). The long Stokes shifts of CPs can be also leveraged for the 2Ex-3Ch scheme in combination with two standard organic dyes absorbing and emitting in two different channels.

FIG. 3 presents, in FIG. 3A, the expected optical properties of a fluorophore set used for 1Ex-2Ch detection. The fluorophores should show similar excitation spectra (dotted line) and allow excitation at a single wavelength. A long Stokes shift fluorophore and a short/standard Stokes shift fluorophore allows emission across two different channels, while a medium Stokes shift fluorophore with broad emission in both channels can act as the “corner” ffN. In FIGS. 3B and C, examples of options to introduce CPs for 1Ex-2Ch detection are illustrated.

Another labeling option is introducing CPs as a medium Stokes shift fluorophore for the “corner” base (ffC, FIG. 3C). As broad emission profiles that can span across two channels are more prevalent among CPs than small molecule organic dyes, it is expected that CPs could act as brighter fluorophores for the “corner” cloud, which would allow “undimming” of the other FFNs and therefore higher signal/SNR.

In reference to FIGS. 3A-3C, some embodiments can use nucleosides comprising the first type nucleobase are labeled with a blue-blue fluorophore (here, adenosine (A)), nucleosides comprising the second type nucleobase are labeled with a blue-green fluorophore (here, cytosine (C)) having the long Stokes shift CPs disclosed herein, the third-type nucleobase are labeled with the blue-blue fluorophore and the blue-green fluorophore (the medium Stokes shift, broad emission CPs) or the third-type nucleobase can be labeled with a fluorophore having an emission between blue emission of the first-type nucleobase and the green emission of the second type nucleobase, and the nucleosides with the fourth-type nucleobase is unlabeled. The later detected by a lack of fluorescent signal.

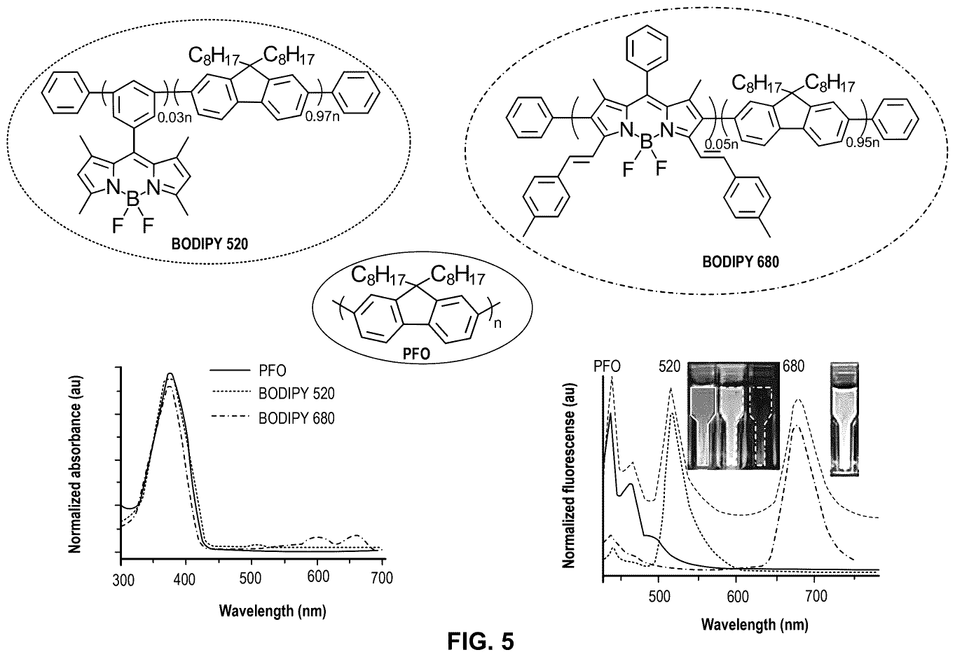

Further, long Stokes shift CPs can be used in combination with standard, short Stokes shift dyes and medium Stokes shift dyes for a 1Ex-3Ch detection scheme if these fluorophores share a common excitation range as illustrated in FIG. 4. In this regard, FIG. 4 shows, in FIG. 4A, an example of expected optical properties of a fluorophore set used for 1Ex-3Ch detection. The fluorophores should show similar excitation spectra (dotted line) and allow excitation at a single wavelength and show different Stokes shifts that allow emission (solid line) across three different channels. In FIG. 4B, examples of options to introduce CPs for 1Ex-3Ch detection are presented.

Although it is believed that, in some situations, a combination of long Stokes shift CPs and standard organic dyes with short/medium Stokes shift is more easily implemented, a full FFN set labeled with CPs can be also designed. This may be much more challenging due to the broad emission profiles of CPs and crosstalk between different channels.

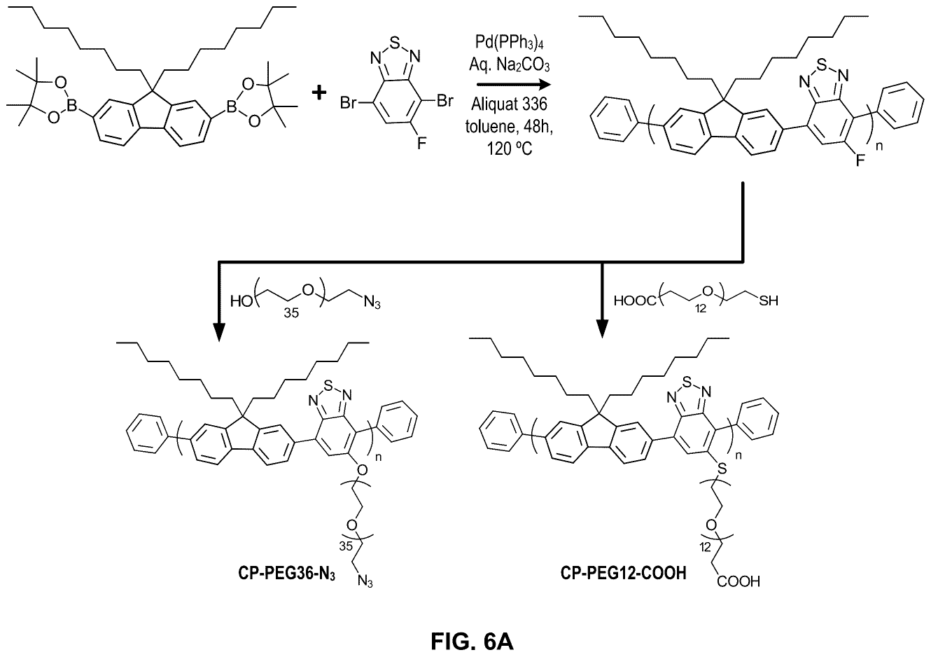

One approach to allow a full CP ffN set is to design donor-acceptor conjugated polymer backbones, where the acceptor is a narrow emissive dye (e.g., BODIPY). By incorporating small percentages of the acceptor dye, the material can retain the excitation properties of the donor and emit at different wavelengths depending on the nature of the dye, allowing different effective Stokes shifts. It is believed that this approach could still benefit from the attractive photophysical properties of conjugated polymers and lead to higher emission intensity for the dye. Such polymers with a common excitation range and narrow emission across three different channels have been previously synthesized in literature using a polyfluorene backbone and different BODIPY dyes as illustrated in FIG. 5. With different donor and acceptor units, the optical properties of these materials could be optimized to match the requirements of Illumina platforms. In this regard, FIG. 5 shows a literature example of 1Ex-3Ch conjugated polymers with narrow emission profiles using BODIPY acceptor dyes. See, Anal. Chem. 2017, 89, 6232-6238.

### 1Ex-2Ch Base Detection Using CPNs

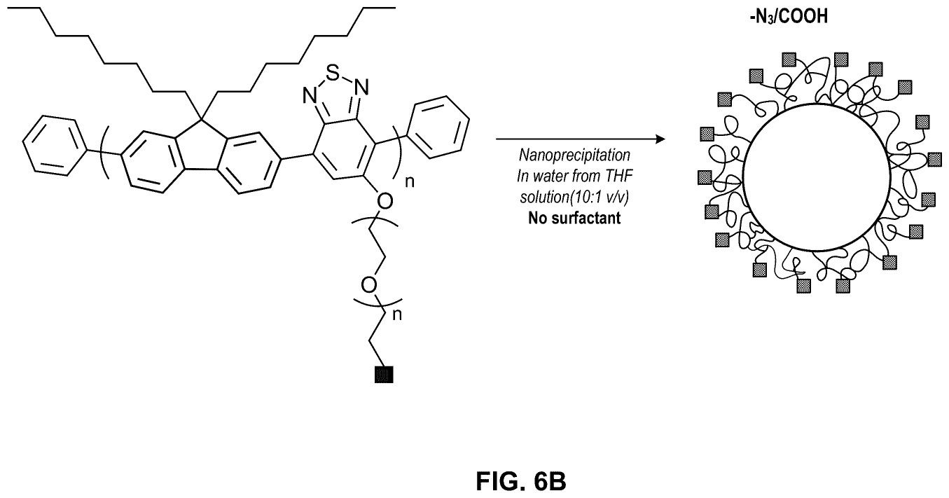

To demonstrate the concept, a poly(5,6-difluoro-2,1,3-benzothiadiazole-alt-9,9-dioctyl-fluorene) backbone was chosen due to its high brightness in aqueous media in nanoparticle form. A fluorinated derivative (CP-F, FIG. 6A) was synthesized by Suzuki polymerization to allow introduction of PIL functionality as a side chain via nucleophilic aromatic substitution. Azide functionality was introduced by reacting CP-F with azido-PEG36-alcohol in the presence of base, giving CP-PEG36-N3 (FIG. 6B). The polymer was used to form CPNs using a nanoprecipitation method, giving self-stabilized particles in water. Similarly, CP-PEG12-COOH was used to form CPNs with carboxylic acid functionality, which was used to label with Streptavidin using NHS/EDC coupling.

FIG. 6 illustrates, in FIG. 6A, the synthesis of CP-F, CP-PEG36-N3 and CP-PEG12-COOH. In FIG. 6B, formation of functionalized CPNs from the conjugated polymers is illustrated. The conjugated polymer can be dissolved in tetrahydrofuran and rapidly injected to water, followed by sonication for 3 min.

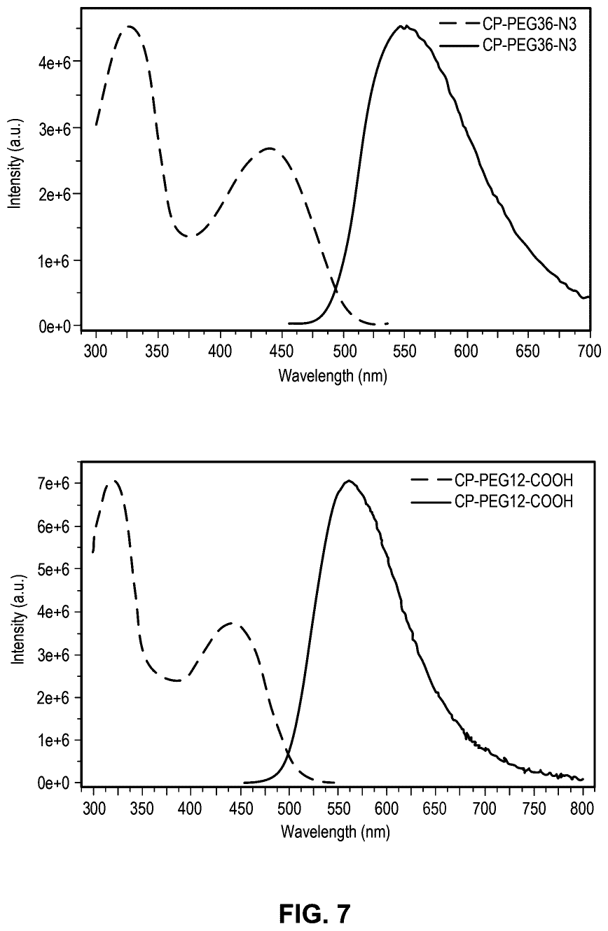

The excitation and emission spectra of the two polymers are shown in FIG. 7. CP-PEG36-N3 and CP-PEG12-COOH nanoparticles show an excitation peak at 445 and 443 nm, respectively, and emission at λ>500 nm, indicating compatibility with blue excitation lasers and green emission filters therefore 1Ex-2Ch base detection.

FIG. 7 shows excitation (dotted line) and emission (solid line) spectra of CP-PEG36-N3 (left) and CP-PEG12-COOH (right) nanoparticles in water. The fluorescence intensity of CP-PEG36-N3 nanoparticles (N-average˜20 nm by DLS) was measured for a range of concentrations in scan mix relative to the Illumina green dye, NR550-C4-COOH. Normalizing by concentration, the CPNs show ˜1400× higher fluorescence intensity in solution compared to the organic dye and therefore great potential to act as bright fluorophores for SBS.

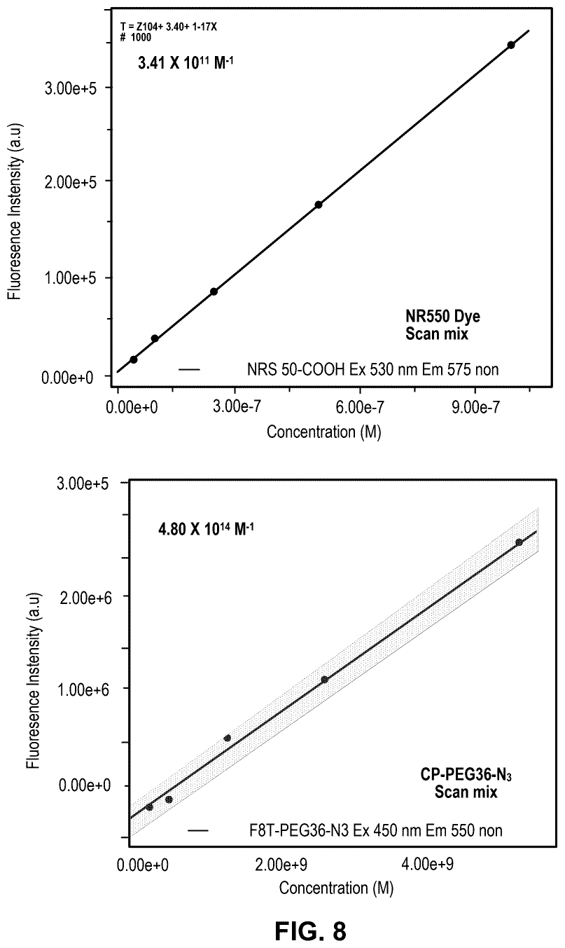

FIG. 8 presents fluorescence intensity vs. concentration for NR550-C4-COOH (left) and CP-PEG36-N3 nanoparticles (right) in scan mix. The concentration of nanoparticles was determined based on solid content and DLS N-average of the particles, assuming spherical particles and a particle density of 1 g/cm³.

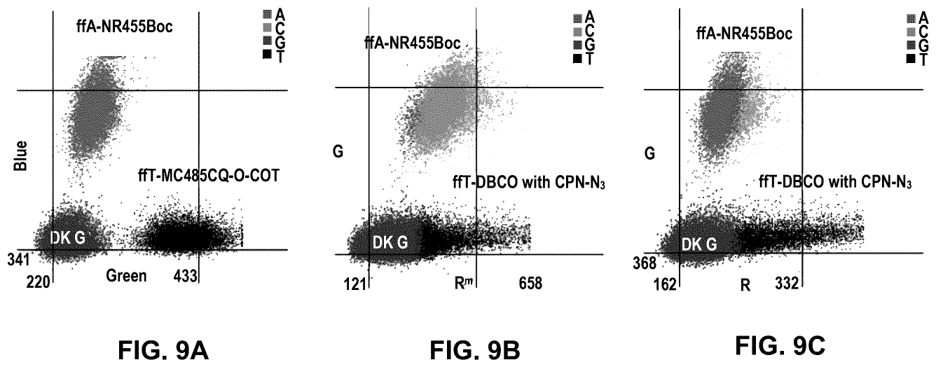

To test the compatibility of CP-PEG36-N3 with 1Ex-2Ch, SBS runs were conducted o with a blue laser operating at 448 nm and images were taken simultaneously through collection channels which are in blue (470-507 nm) and green (583-660 nm). FIG. 9A illustrates the scatterplot obtained with standard ffN incorporation using a ffT labeled with an Illumina green-emitting long Stokes shift dye, MC485CQ-O-COT, which acts as the control. The incorporation mix was modified by substituting ffT-MC485CQ-O-COT with an unlabeled ffT having a reactant DBCO moiety and post-incorporation labeling was attempted with the azide nanoparticles. After allowing to bind for either 60 s (FIG. 9B) or 300 s (FIG. 9C), formation of a cloud in the green channel was observed, indicating successful base labeling using the CPNs. FIG. 9 presents scatterplots obtained with a) a standard incorporation mixture containing FFG, FFC-G2-NR550S0, FFT-G2-MC485CQ-O-COT and FFA-G2-BL-NR455Boc; b) and c) a second incorporation mix containing FFG, ffC-G2-NR550S0, FFA-G2-BL-NR455Boc and an unlabeled FFT having a reactant DBCO moiety. The post-incorporation reagent contains azide-functionalized conjugated polymer nanoparticles (CP-PEG36-N3) dispersed in HT1 hybridization buffer. The post-incorporation mixture was introduced to incorporated ffNs and allowed to bind for b) 60 s and c) 300 s at 60° C. The experiments were performed using a P2 flowcell clustered on board with PhiX (1 nM), with 5 cycles recorded for each condition.

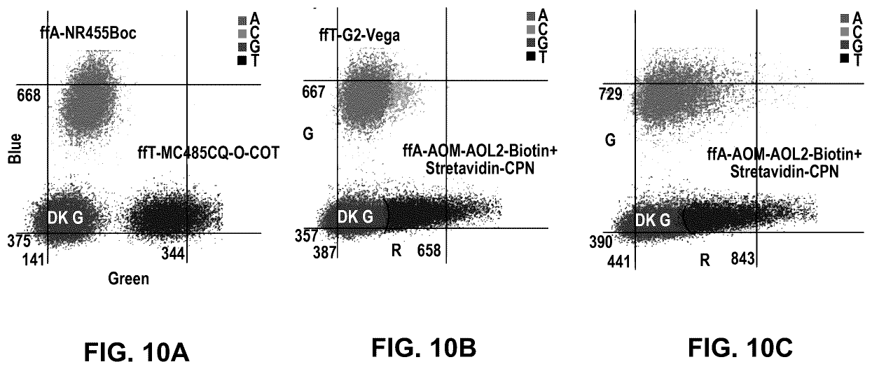

It was predicted that more efficient labeling and higher signal could be obtained by using labeling chemistry with faster kinetics than the azide-DBCO click reaction. Non-covalent streptavidin-biotin labeling was chosen due to the high affinity and stability of the interaction, even for the low concentrations obtained with the nanoprecipitation method described above (0.020-2 μM). CP-PEG12-COOH was functionalized with Streptavidin using NHS/EDC coupling and tested on NextSeq2000, with the resulting scatterplots shown in FIG. 10. The use of biotin-streptavidin chemistry allowed fast labeling of the incorporated base (ffA), and green channel signal was observed after only 1 s of incubation at 60° C.

FIG. 10 presents NextSeq 2000 scatterplots obtained with a) a standard incorporation mixture containing FFG, FFC-G2-NR550S0, FFT-G2-MC485CQ-O-COT and FFA-G2-BL-NR455Boc; b) and c) a second incorporation mix containing FFG, FFC-G2-NR550S0, FFT-G2-Vega and an unlabeled FFA having a reactant biotin moiety.

The post-incorporation reagent contains streptavidin-functionalized conjugated polymer nanoparticles dispersed in Illumina binding buffer. The post-incorporation mixture was introduced to incorporated ffNs and allowed to bind for b) 1 s and c) 60 s at 60° C. The instant results demonstrate the potential of conjugated polymer nanoparticles to act as long Stokes shift fluorophores for 1Ex-2Ch detection and their compatibility with the SBS workflow. Combined with their enhanced brightness in solution and photostability, such materials could bring great benefits for SNR and data quality in future platforms and would expand the library of base detection fluorophores beyond small molecule organic dyes.

1. A method of fluorescently detecting a polynucleotide material, the method comprising
providing a substantially monoclonal polynucleotide analyte immobilized on a solid support surface;
contacting the analyte with a one-exposure, two channel reagent solution, wherein the reagent solution comprises a mixture of nucleosides, each nucleoside comprising a first-type, second-type, third-type or fourth-type nucleobases, a polymerase, and a primer,
wherein nucleosides comprising the first type nucleobase are labeled with a blue-blue fluorophore, nucleosides comprising the second type nucleobase are labeled with a blue-green fluorophore having the following structure: 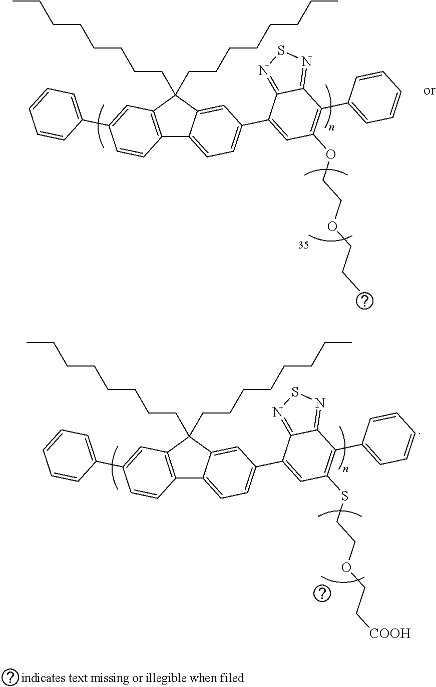
wherein n is 1 to about 1000;
wherein the conjugated polymer nanoparticle has a Stokes shift of at least 90 m,
wherein nucleosides with the third-type nucleobase are labeled with the blue-blue fluorophore and the blue-green fluorophore or the third-type nucleobase can be labeled with a fluorophore having an emission between blue emission of the first-type nucleobase and the green emission of the second type nucleobase, and the nucleosides with the fourth-type nucleobase is unlabeled; and
wherein one of the nucleosides is configured to hybridize to the analyte;
exposing the analyte to excitation in a blue channel; and
detecting a fluorescent signal in the analyte;
wherein a blue channel signal indicates a first-type nucleoside hybridized to the analyte, a green channel signal indicates a second-type nucleoside, a signal detectable in both the blue and green channels indicates a third-type nucleoside, and no signal indicates a fourth-type nucleoside.

2. The method of claim 1, wherein the Stokes shift is at least 100 nm.

3. The method of claim 1, wherein the first, second, third and fourth-type nucleosides are selected from adenosine (A), cytosine (C), guanosine (G) and thymidine (T) such that each of the first, second, third and fourth-type nucleosides are a different nucleoside.

4. The method of claim 1, wherein the first, second, third and fourth-type nucleosides are selected from adenosine (A), cytosine (C), guanosine (G) and uracil (U) such that each of the first, second, third and fourth-type nucleosides are a different nucleoside.

5. The method of claim 1, wherein the solid support surface comprises
a base layer,
an optic and chemical support layer of cured resin material adhered to the support layer, wherein the optic and chemical support layer comprises an array separated by interstitial regions of featureless resin surface, each array comprising:
a plurality of nanowells impressed in the optic and chemical support layer, wherein the nanowells define openings coplanar with the featureless resin surface, the nanowells comprising:
a base portion providing a solid support for optic detection of the polynucleotide analyte,
a reaction site collocated with at least a portion of the solid support,
a conformal shell of high refractive index medium supporting the interior volume of the cavity, and
wherein the surface chemistry is localized to the reaction site and comprises a capture agent configured to interact with a constituent analyte of the polynucleotide material.

6. The method of claim 1, wherein n is 1 to about 10.

7. The method of claim 1, wherein the Stokes shift is about 100 nm to about 120 nm.

8. The method of claim 1, wherein the blue-green fluorophore is a nanoparticle.

9. A method of fluorescently detecting a polynucleotide material, the method comprising
providing a substantially monoclonal polynucleotide analyte immobilized on a solid support surface;
contacting the analyte with a one-exposure, two channel reagent solution, wherein the reagent solution comprises a mixture of nucleosides, each nucleoside comprising a first-type, second-type, third-type or fourth-type nucleobases, a polymerase, and a primer,
wherein nucleosides comprising the first type nucleobase are labeled with a blue-blue fluorophore, nucleosides comprising the second type nucleobase are labeled with a blue-green fluorophore having a Stokes shift of at least 90 nm,
wherein nucleosides with the third-type nucleobase are labeled with the blue-blue fluorophore and the blue-green fluorophore or the third-type nucleobase can be labeled with a fluorophore having an emission between blue emission of the first-type nucleobase and the green emission of the second type nucleobase, and the nucleosides with the fourth-type nucleobase is unlabeled; and
wherein one of the nucleosides is configured to hybridize to the analyte;
exposing the analyte to excitation in a blue channel; and
detecting a fluorescent signal in the analyte;
wherein a blue channel signal indicates a first-type nucleoside hybridized to the analyte, a green channel signal indicates a second-type nucleoside, a signal detectable in both the blue and green channels indicates a third-type nucleoside, and no signal indicates a fourth-type nucleoside.

10. The method of claim 9, wherein the Stokes shift is at least 100 nm.

11. The method of claim 9, wherein the first, second, third and fourth-type nucleosides are selected from adenosine (A), cytosine (C), guanosine (G) and thymidine (T) such that each of the first, second, third and fourth-type nucleosides are a different nucleoside.

12. The method of claim 9, wherein the first, second, third and fourth-type nucleosides are selected from adenosine (A), cytosine (C), guanosine (G) and uracil (U) such that each of the first, second, third and fourth-type nucleosides are a different nucleoside.

13. The method of claim 9, wherein the solid support surface comprises
a base layer,
an optic and chemical support layer of cured resin material adhered to the support layer, wherein the optic and chemical support layer comprises an array separated by interstitial regions of featureless resin surface, each array comprising:
a plurality of nanowells impressed in the optic and chemical support layer, wherein the nanowells define openings coplanar with the featureless resin surface, the nanowells comprising:
a base portion providing a solid support for optic detection of the polynucleotide analyte,
a reaction site collocated with at least a portion of the solid support,
a conformal shell of high refractive index medium supporting the interior volume of the cavity, and
wherein the surface chemistry is localized to the reaction site and comprises a capture agent configured to interact with a constituent analyte of the polynucleotide material.

14. The method of claim 9, wherein the Stokes shift is about 100 nm to about 120 nm.

15. The method of claim 9, wherein the blue-green fluorophore is a nanoparticle.

16. A one exposure, two channel reagent kit comprising:
a mixture of nucleosides in solution, each nucleoside comprising a first-type, second-type, third-type or fourth-type nucleobase;
a polymerase; and
a primer;
wherein nucleosides comprising the first type nucleobase are labeled with a blue-blue fluorophore, nucleosides comprising the second type nucleobase are labeled with a blue-green chromophore having a Stokes shift of at least 90 nm,
nucleosides with the third-type nucleobase are labeled with the blue-blue fluorophore and the blue-green fluorophore or the third-type nucleobase can be labeled with a fluorophore having an emission between blue emission of the first-type nucleobase and the green emission of the second type nucleobase, and the nucleosides with the fourth-type nucleobase is unlabeled.

17. The method of claim 16, wherein the blue-green fluorophore is a nanoparticle.

18. The method of claim 16, wherein the Stokes shift is at least 100 nm.

19. The method of claim 16, wherein the first, second, third and fourth-type nucleosides are selected from adenosine (A), cytosine (C), guanosine (G) and thymidine (T) such that each of the first, second, third and fourth-type nucleosides are a different nucleoside.

20. The method of claim 16, wherein the first, second, third and fourth-type nucleosides are selected from adenosine (A), cytosine (C), guanosine (G) and uracil (U) such that each of the first, second, third and fourth-type nucleosides are a different nucleoside.
